(19) United States
(12) Patent Application Publication
(10) Pub. No.: US 2013/0274114 A1
(43) Pub. Date: Oct. 17, 2013

(54) MULTIPOTENT STEM CELL-BASED CULTURE SYSTEMS AND MODELS

(71) Applicant: LEONARD SCIORRA, NESHANIC STATION, NJ (US)

(72) Inventor: LEONARD SCIORRA, NESHANIC STATION, NJ (US)

(73) Assignee: SAINT PETER'S UNIVERSITY, JERSEY CITY, NJ (US)

(21) Appl. No.: 13/630,512

(22) Filed: Sep. 28, 2012

Related U.S. Application Data
(60) Provisional application No. 61/540,507, filed on Sep. 28, 2011.

(51) Int. Cl. C12N 5/071

(52) U.S. Cl. CPC C12N 5/0625
USPC 506/2; 435/371; 435/370; 435/368; 435/366

(57) ABSTRACT

This invention generally relates to multipotent stem cell-based research tools. More particularly, the present invention relates to culture systems and 3-dimensional tissue models that may be used for identifying agents useful for treating diseases and conditions and that are suitable for high throughput screening applications. This present invention is based, in part, on the discovery of a method for propagating multipotent stem cells from human skin fibroblasts and subsequently differentiating those multipotent stem cells into cells of any of the three germ layers. Aspects of the invention include drug discovery tools as a high throughput screen; 3-dimensional tissue engineering model, and drug discovery tools thereof; research tools for identifying genes that are important for acquiring multipotency and for identifying genes that are important for lineage-specific differentiation, and drug discovery tools thereof; diagnostic tools for identifying defective genes; and autologous therapies based on the propagated multipotent stem cells.

### PRIORITY CLAIM

This application claims priority to U.S. Provisional Patent Application No. 61/540,507, filed Sep. 28, 2011, the entirety of which is hereby incorporated by reference.

### BACKGROUND

This invention generally relates to multipotent stem cell-based research tools. More particularly, the present invention relates to culture systems and 3-dimensional tissue models that may be used for identifying agents useful for treating diseases and conditions and that are suitable for high-throughput screening applications. Additionally, the present invention is directed to identifying genes that are involved in the process of acquisition of multipotency. The present invention is also directed to identifying genes that are involved in the process of lineage-specific differentiation. The present invention is based, in part, on the discovery of methods for propagation of multipotent stem cells from human skin fibroblast samples as is disclosed in International Patent Publication No. WO 2009/151844, which is incorporated herein by reference in its entirety.

International Patent Publication No. WO 2009/151844 describes methods for propagating multipotent stem cells from human skin fibroblast samples. These multipotent stem cells were then subsequently differentiated into cells of any of the three germ layers, including adipose, hepatic, muscle, and nerve cells.

The invention described in International Patent Publication No. WO 2009/151844 further provides that the multipotent stem cells could be propagated and differentiated to be used for regeneration, recreation repopulation and/or reconstitution of desired tissues and organs. For example, International Patent Publication No. WO 2009/151844 provides for autologous therapies based on the propagated multipotent stem cells for regeneration of tissues, for use as grafts, tissue/organ replacement or supplementation.

### DESCRIPTION OF THE EMBODIMENTS

This invention generally relates to multipotent stem cell-based research tools. More particularly, the present invention relates to culture systems and 3-dimensional tissue models that may be used for identifying agents useful for treating diseases and conditions and that are suitable for high-throughput screening applications. Additionally, the present invention is directed to identifying genes that are involved in the process of acquisition of multipotency. The present invention is also directed to identifying genes that are involved in the process of lineage-specific differentiation. The present invention is based, in part, on the discovery of methods for propagation of multipotent stem cells from human skin fibroblast samples as is disclosed in International Patent Publication No. WO 2009/151844, which is incorporated herein by reference in its entirety.

To date, much of the work on stem cells has entailed obtaining stem cells from embryonic sources. However this is often accompanied by moral and ethical issues, as this typically involves the destruction of an embryo. Accordingly, scientists have sought alternative and uncontroversial means of acquiring stem cells. Some examples include using adult stem cells, amniotic stem cells, or induced pluripotent stem cells. Adult stem cells, also known as somatic stem cells, are found in adult tissues throughout the body. Amniotic stem cells are of mesenchymal origin extracted from amniotic fluid. Induced pluripotent stem cells are artificially derived, typically by taking an adult somatic cell and inducing pluripotency by forcing expression of specific genes, for example, by recombinant gene or protein transfer. Although each of these means is uncontroversial, they are not without drawbacks. For example, it is often difficult to get large numbers of stem cells and acquiring such cells may often require selecting and isolating out rare stem cells against a backdrop of non-stem cells. Moreover, induced pluripotent stem cells may result in the unwanted induction of genes, which may be oncogeneic. Therefore, there was a need in the art for an uncontroversial method for obtaining a large number of multipotent stem cells without the need for isolation or transfer of recombinant gene or protein.

International Patent Publication No. WO 2009/151844 describes methods for propagating, without the need for an initial isolation or for gene or viral transduction, multipotent stem cells from human skin fibroblasts of both sexes, of all races, using selective culture conditions. Selective culture conditions may consist of an appropriate medium comprising amniotic growth fluid media (AFM) and other media and various growth factors (as described in DeCoppi et al., comprising α-MEM (Invitrogen), 15% ES-FBS (Invitrogen), 1% L-Glutamine, and 1% Pen/Strep, supplemented with 18% CHANG MEDIUM® B (Irvine Scientific) and 2% CHANG MEDIUM® C (Irvine Scientific)). The AFM comprises α-MEM media plus supplements. These multipotent stem cells may then be subsequently differentiated into cells of any of the three germ layers, including adipose, hepatic, muscle, and nerve cells. That is, these methods provide a relatively simple tissue culturing procedure to take cells—(frozen or otherwise) obtained from individuals of all ages—and grown them to a point whereby large numbers of multipotent cells can be reproducibly obtained in culture at various scales and subsequently differentiated along cell lineages that resemble cells of any of the three germ layers, including nerve, adipose, hepatic, and muscle cells. The methods underlying the invention provide an advantageous alternative to methods of attaining/obtaining embryonic stem cells, adult stem cells, amniotic stem cells, or induced pluripotent stem cells. Even skin fibroblast cells that were from passages 8-10 were able to propagate substantial numbers of multipotent stem cells. Using these methods, after 3 passages, large numbers of cells that were CD117⁺ and/or NANOG⁺ were observed. Both CD117 and NANOG are stem cell markers well known in the art. There was, however, an observed inverse relationship with age of the patient and number of CD117⁺ cells.

Microarray studies were conducted to measure the differential expression of the genes that are either up- or down-regulated upon transfer of the skin fibroblast cells (in an Eagles-based MEM media) into media that promotes the acquisition of multipotency (α-MEM media plus supplements). These studies show that once the skin fibroblasts cells are transferred to the culture media that promotes propagation of multipotent stem cells, the cells undergo a complex change in the gene pattern of expression involving numerous genes.

Once the multipotent stem cells are propagated, these cells may then subject to differentiation into cells of the 3 germ layers. The setting and culture conditions that promote any given lineage-specific differentiation are well known in the art. For example, multipotent stem cells may be subject to differentiation in standard tissue culture conditions, growing in a monolayer. However, in addition, the setting and culture conditions may be such that the multipotent stem cells may be encouraged to grow and differentiate 3-dimensionally onto a scaffold or a matrix, such as a plate with laminin-coated beads. This 3-dimensional tissue model provides setting and conditions for differentiation that more closely proximate the tissues/cells in their actual in vivo environment.

In addition, it is possible to conduct studies to identify and examine the differential gene expression, i.e., which genes are up- or down-regulated, during the lineage-specific differentiation process. For example, microarray analyses may be conducted at various time points during lineage-specific differentiation to examine gene expression patterns during that lineage-specific differentiation. These studies allow for the observation of how gene expression changes from the initiation of differentiation to the generation of each of the lineage-specific tissues. Such analysis may identify genes that are important in the function and/or development of that cell lineage. It is possible that, in the case of a 3-dimensional tissue model, microarray data may represent an improvement over microarray data generated by measuring the differential expression of genes during the process of lineage-specific differentiation when done under conventional means under selective culture conditions. That is, the microarray data generated from a 3-dimensional engineering model might be more accurate, since it more closely proximates the tissues/cells in their actual in vivo environment.

Neither chromosome nor Comparative Genomic Hybridization studies showed any anomalies. That is, based on studies thus far, no obvious chromosome aberrations were observed in multipotent stem cells or cells derived from multipotent stem cells generated by the methods described.

There are a plurality of setting and culture conditions for any given lineage-specific differentiation. By way of example, provided below are setting and culture conditions for adipogenic, hepatic, myogenic, and neurogenic differentiation. These examples are meant to be illustrative only and not limiting.

Adipogenic Differentiation:

Cells were seeded at a density of 3,000 cells/cm² onto chamber slides (Nunc). They were cultured in DMEM low-glucose medium (Sigma-Aldrich) with 10% FBS (Invitrogen), 1% Pen/Strep, and the following adipogenic supplements: 1 μM dexamethasone (Sigma-Aldrich), 1 mM 3-isobutyl-1-methylxanthine (Sigma-Aldrich), 10 μg/ml insulin (Sigma Aldrich), and 60 μM indomethacin (Sigma-Aldrich). Cells were maintained in adipogenic differentiation media for up to 20 days.

Hepatic Differentiation:

Cells were seeded at a density of 5,000 cells/cm² onto chamber slides coated with Matrigel (Sigma-Aldrich). The cells were first expanded for 3 days in AFM then placed in hepatic differentiation media: DMEM low-glucose with 15% FBS, 300 μM monothioglycerol (Sigma-Aldrich), 20 ng/ml hepatocyte growth factor (Sigma-Aldrich), 10 ng/ml oncostatin M (Sigma-Aldrich), 10-7 M dexamethasone (Sigma-Aldrich), 100 ng/ml FGF4 (Peprotech), 1xITS (Invitrogen) and 1% Pen/Strep. The cells were maintained in this differentiation medium for 17 days, with medium changes every third day.

Myogenic Differentiation:

Cells were seeded at a density of 3,000 cells/cm² onto chamber slides coated with Matrigel and grown in DMEM low-glucose with 10% horse serum (Invitrogen), 0.5% chick embryo extract, and 1% Pen/Strep. Twelve hours after seeding, 3 μM 5-aza-2′-deoxycytodine (5-azaC; Sigma-Aldrich) was added to the culture medium for 24 hours. Incubation continued in complete medium lacking 5-azaC, with medium changes every 3 days. Cells were maintained in myogenic differentiation media for up to 20 days.

Neurogenic Differentiation:

Cells were seeded at a concentration of 3,000 cells/cm² onto either chamber slides or Nunc 6 well Petri dishes for micro array studies. These cells were cultured in DMEM/F12 media (Invitrogen), supplemented with 200 uM BHA (Sigma-Aldrich), N2 (Invitrogen), 25 ng/ml NGF (Invitrogen), 10 ng/ml bFGF (Invitrogen) 15% ESFBS, 1% Pen/Strep and 1% L-Glutamine. Every two days an additional 25 ng/ml of NGF and 10 ng/ml of bFGF were added to the cultures. After 6 or 7 days the cultures were examined and photographed for nerve morphology or harvested for microarray analysis. The medium used in this neurogenic differentiation media contains no DMSO. A second set of experiments was set up using the above media but lacking DMSO and BHA

In one aspect, the invention may be used as a research tool for drug discovery, e.g., in a high throughput drug screen. This high throughput drug screen would provide a means to test the efficacy of a plurality of compounds or substances on tissues/cells, including a patient's own tissues/cells. In a further aspect, a high throughput drug screen may entail administration of compound libraries to plates harboring differentiated fibroblast-derived lineage-specific cells. Lineage-specific, in this context, refers to cells of any type, including, but not limited to, cells of the integumentary system, nervous system, teeth, nervous system, eyes, digestive system (stomach, intestine, gallbladder, exocrine pancreas), endocrine, respiratory, liver, urogenital, cartilage/bone/muscle, urinary, reproductive system, blood system, immune system, circulatory system. In yet a further aspect, the high throughput drug screen is applied to lineage-specific cells from a patient suffering from a disease or condition affecting cells of that lineage in vivo. Furthermore, large amounts of differentiated fibroblast-derived lineage-specific cells may be generated for use in the high throughput drug screen by employing the methods described in International Patent Publication No. WO 2009/151844, which methods, including everything else in International Patent Publication No. WO 2009/151844 are incorporated herein by reference. One major advantage of this aspect of the invention is the ability to determine if a patient's tissues/cells respond to a battery of potential drug compounds or biological substances, in some cases, without the need to invasively obtain those tissues from the patient suffering from a disease or condition. Some diseases or conditions that may be useful for the high throughput drug screen include, but are not limited to, Alzheimer's Disease, Amyotrophic Lateral Sclerosis, Parkinson's Disease, Liver Sclerosis, Heart Failure, Type 1 Diabetes, and Muscular Dystrophy.

In one aspect, the invention comprises a stem-cell based culture system that may be used for generating a 3-dimensional tissue engineering model. This may be done by altering the culturing methods described in International Patent Publication No. WO 2009/151844 such that the cells propagate in a culture setting that will foster 3-dimensional tissue growth, such as with a scaffold or matrix. Such methods are well known to one of ordinary skill in the art. One such example of an appropriate scaffold or matrix is a plate coated with laminin-coated beads. For example, the multipotent stem cells may be encouraged to grow and differentiate 3-dimensionally onto a scaffold or a matrix, such as a plate with laminin-coated beads. Other examples of appropriate scaffolds or matrices are microfluidic chambers and nanofiber membrane scaffolds. This 3-dimensional tissue model provides setting and conditions for differentiation that more closely proximate the tissues/cells in their actual in vivo environment. Moreover, in this capacity, the 3-dimensional tissue model may be used for drug discovery—to determine the efficacy of a drug or agent on an individual's patient's tissues/cells in the context of a 3-dimensional tissue engineering model. Some diseases or conditions that may be useful for drug discovery using a 3-dimensional tissue engineering model include, but are not limited to, Alzheimer's Disease, Amyotrophic Lateral Sclerosis, Parkinson's Disease, Liver Sclerosis, Heart Failure, Type 1 Diabetes, and Muscular Dystrophy.

In another aspect, the invention comprises a research tool to identify the function of genes involved in the acquisition of multipotency. Microarray analysis of the fibroblast cells during the propagation stage may reveal the identity of genes whose expression levels change during the process of acquiring multipotency. Accordingly, such analysis may reveal one or more genes that are important for the process of generating multipotent stem cells. This information may be useful as a research tool for developing new and different strategies for obtaining and generating large amounts of multipotent stem cells derived from adult tissues/cells. The genes identified may be useful to treat diseases and conditions such as Alzheimer's Disease, Amyotrophic Lateral Sclerosis, Parkinson's Disease, Liver Sclerosis, Heart Failure, Type 1 Diabetes, and Muscular Dystrophy.

In another aspect, the invention comprises a research tool to identify the genes involved in the process of lineage-specific differentiation. Microarray analysis of the cells during the differentiation process reveals the identity of genes whose expression levels change during and as a result of lineage-specific differentiation. Identification of such genes maybe useful in various applications. For example, identification of such genes may reveal genes important for development of lineage-specific cells. Additionally, genes important in the lineage-specific development of a particular cell type may also function as suitable targets for therapeutic intervention to treat diseases and conditions affecting that specific cell type. The genes identified may be useful to treat diseases and conditions such as Alzheimer's Disease, Amyotrophic Lateral Sclerosis, Parkinson's Disease, Liver Sclerosis, Heart Failure, Type 1 Diabetes, and Muscular Dystrophy. In a related aspect, the lineage-specific differentiation may be in a 3-dimensional tissue model. That is, the multipotent stem cells may be encouraged to grow and differentiate 3-dimensionally onto a scaffold or a matrix, such as a plate with laminin-coated beads. This 3-dimensional tissue model provides setting and conditions for differentiation that more closely proximate the tissues/cells in their actual in vivo environment. In this way, it is possible that the microarray data generated by measuring the differential expression of genes during the process of lineage-specific differentiation in a 3-dimensional tissue modeling may represent an improvement over microarray data generated by measuring the differential expression of genes during the process of lineage-specific differentiation when done under conventional means under selective culture conditions. That is, the microarray data generated from a 3-dimensional engineering model might be more accurate, since it more closely proximate the tissues/cells in their actual in vivo environment.

In another aspect, the invention comprises a diagnostic tool for identifying one or more genes that may be defective in an individual. Microarray analysis of the cells during the differentiation process reveals the identity of genes whose expression levels change during and as a result of lineage-specific differentiation. The differentiation process may be in a 3-dimensional tissue model. That is, the multipotent stem cells may be encouraged to grow and differentiate 3-dimensionally onto a scaffold or a matrix, such as a plate with laminin-coated beads. This 3-dimensional tissue model provides setting and conditions for differentiation that more closely proximate the tissues/cells in their actual in vivo environment. If microarray analysis reveals that one or more genes are differentially expressed in fibroblast-derived lineage-specific cells taken from an individual suffering from a disease or condition as compared to a normal individual, this may indicate that the expression of those one or more genes is altered, mutated, or defective in that individual. In this way this culture system may function as a diagnostic tool to identify underlying genetic causes of diseases or conditions. The genes identified may be useful to identify diseases and conditions such as Alzheimer's Disease, Amyotrophic Lateral Sclerosis, Parkinson's Disease, Liver Sclerosis, Heart Failure, Type 1 Diabetes, and Muscular Dystrophy.

In another aspect, the invention provides for autologous therapies based on the propagated multipotent stem cells for regeneration of tissues, for use as grafts, tissue/organ replacement or supplementation, as described in International Patent Publication No. WO 2009/151844. One advantage of the methods described in International Patent Publication No. WO 2009/151844, is that fibroblast-derived multipotent stem cells are propagated without the need for recombinant gene or protein transfer, rendering the multipotent stem cells of the invention safer for use in autologous therapy as compared to other methods that employ recombinant gene or protein transfer. Moreover, karyotype and comparative genomic hybridization (CGH) studies described in Example 6 reveal that the fibroblast-derived multipotent stem cells described herein and in International Patent Publication No. WO 2009/151844 do not exhibit elevated levels of mutations, suggesting the safety of the multipotent stem cells for various applications, such as for autologous therapies. The autologous therapies may be useful to treat disease and conditions such as such as Alzheimer's Disease, Amyotrophic Lateral Sclerosis, Parkinson's Disease, Liver Sclerosis, Heart Failure, Type 1 Diabetes, and Muscular Dystrophy.

In one aspect, the invention provides for a method of generating a 3-dimensional tissue engineering model comprising the steps of: (a) propagating multipotent stem cells from human skin fibroblast culture by growing the cells in a culture containing amniotic fluid growth medium (AFM) and allowing the cells to propagate for at least 3 passages; and (b) subjecting said multipotent stem cells to lineage-specific differentiation by culturing said multipotent stem cells in cells in a culture setting that will foster 3-dimensional tissue growth, such as a scaffold or matrix. In a related aspect, the culture further comprises Embryonic Cell Qualified Fetal Bovine Serum (ES-FBS). In another aspect, the cells are subject to at least 3, 4, 5, 6, 7, or 8 passages in culture. In a related aspect, the method comprises the step of determining the number of multipotent stem cells in the culture, and, in another aspect, the number of CD117⁺ multipotent stem cells in the culture can be determined after each passage. In another aspect, the human skin fibroblast culture is prolonged by continued passages in the culture until a high number of CD117⁺ multipotent stem cells is attained. In a related aspect, the propagated CD117⁺ multipotent stem cells are subject to differentiation when the CD117+ cell count reaches at least about 85%. In another aspect, the propagated cells are cryopreserved after step (a) but before step (b). In another aspect, the propagated multipotent stem cells are capable of differentiating into any of the three germ layers. In a related aspect, the propagated multipotent stem cells are capable of differentiation into adipose, hepatic, muscle, or nerve cells under suitable culture conditions. In yet another aspect, the suitable culture conditions are conditions will foster 3-dimensional tissue growth are culture plates containing laminin-coated beads. In a related aspect, the culture plates containing laminin-coated beads are created by: (a) dissolving laminin in cold phosphate buffer saline (PBS) placed in a tissue culture plate; (b) adding sterile spherical glass beads or a mix of spherical glass beads to the laminin; (c) placing the culture plate in an incubator at 37° C. for at least 12 hours in order to induce polymerization of laminin; and (d) removing excess PBS and allowing the culture plate to completely air dry.

In another aspect, the invention provides a method of generating a 3-dimensional tissue engineering model comprising the steps of: (a) propagating multipotent stem cells from human skin fibroblast culture by growing the cells in a culture containing amniotic fluid growth medium (AFM) and allowing the cells to propagate for at least 3 passages; (b) culturing the multipotent stem cells in the laminin-coated bead plates in a tissue culture media that promotes differentiation into one of the three germ layers, wherein the laminin-coated bead plates were created by: (1) dissolving laminin in cold phosphate buffer saline (PBS) placed in a tissue culture plate; (2) adding sterile spherical glass beads or a mix of spherical glass beads to the laminin; (3) placing the culture plate in an incubator at 37° C. for at least 12 hours in order to induce polymerization of laminin; (4) removing excess PBS and allowing the culture plate to completely air dry; (5) adding the multipotent stem cells to the laminin-coated bead plates; and (6) plating the multipotent stem cells in the laminin-coated bead plates with the multipotent stem cells in an incubator at 37° C.; and (c) subjecting the multipotent stem cells to lineage-specific differentiation under suitable conditions into cells of any of three germ layers.

In a related aspect, the invention provides a method for identifying one or more genes involved in the process of lineage-specific differentiation, said method comprising the steps of: (a) propagating multipotent stem cells from human skin fibroblast culture by growing the cells in a culture containing amniotic fluid growth medium (AFM) and allowing the cells to propagate for at least 3 passages; (b) subjecting said multipotent stem cells to lineage-specific differentiation by culturing said multipotent stem cells under culture conditions suitable for lineage-specific differentiation until differentiated cells result; (c) subjecting said differentiated cells to gene expression profiling using microarray technology; and (d) determining which one or more genes is upregulated or down-regulated during the process of lineage-specific differentiation. In a related aspect, the culture containing amniotic fluid growth medium (AFM) further comprises Embryonic Cell Qualified Fetal Bovine Serum (ES-FBS). In another aspect, the cells are subject to at least 3, 4, 5, 6, 7, or 8 passages in culture. In another aspsect, the method further comprises the step of determining the number of multipotent stem cells in the culture. In another aspect, the number of CD117⁺ multipotent stem cells in the culture can be determined after each passage. In a related aspect, the human skin fibroblast culture is prolonged by continued passages in the culture until a high number of CD117⁺ multipotent stem cells is attained. In a related aspect, the propagated CD117⁺ multipotent stem cells are subject to differentiation when the CD117+ cell count reaches at least about 85%. In another aspect, the propagated cells are cryopreserved after step (a) but before step (b). In another aspect, the propagated multipotent stem cells are capable of differentiating into any of the three germ layers. In a related aspect, the propagated multipotent stem cells are capable of differentiation into adipose, hepatic, muscle, or nerve cells under suitable culture conditions. In another aspect, the suitable culture conditions will foster 3-dimensional tissue growth, such as a scaffold or matrix. In a related aspect, the culture conditions that will foster 3-dimensional tissue growth are culture plates containing laminin-coated beads. In another aspect, the culture plates containing laminin-coated beads are created by: (a) dissolving laminin in cold phosphate buffer saline (PBS) placed in a tissue culture plate; (b) adding sterile spherical glass beads or a mix of spherical glass beads to the laminin; (c) placing the culture plate in an incubator at 37° C. for at least 12 hours in order to induce polymerization of laminin; and (d) removing excess PBS and allowing the culture plate to completely air dry.

1. In one aspect, the invention relates to an isolated multipotent stem cell, or a collection of culture of isolated multipotent stem cells, obtained by a method of propagating multipotent stem cells from human skin fibroblast culture by growing the cells in a culture containing amniotic fluid growth medium (AFM) and allowing the cells to propagate for at least 3 passages. In a related aspect, the culture further comprises Embryonic Cell Qualified Fetal Bovine Serum (ES-FBS). In another aspect, the multipotent stem cells are capable differentiating into any of the three germ layers.

In another aspect, the invention is directed to an isolated differentiated cell, or a collection of culture of isolated differentiated cells, obtained by: (a) propagating multipotent stem cells from human skin fibroblast culture by growing the cells in a culture containing amniotic fluid growth medium (AFM) and allowing the cells to propagate for at least 3 passages; and (b) subjecting said multipotent stem cells to lineage-specific differentiation by culturing said multipotent stem cells under culture conditions suitable for lineage-specific differentiation until differentiated cells result. In a related aspect, the differentiated cells are cells of any of the three germ layers. In another aspect, the cells of any of the three germ layers include adipose, hepatic, muscle, or nerve cells. In a related aspect, the culture conditions suitable for lineage-specific differentiation foster 3-dimensional tissue growth. In another aspect, the culture conditions suitable for lineage-specific differentiation foster 3-dimensional tissue growth are culture plates containing laminin-coated beads. In another aspect, the culture plates containing laminin-coated beads are created by: (a) dissolving laminin in cold phosphate buffer saline (PBS) placed in a tissue culture plate; (b) adding sterile spherical glass beads or a mix of spherical glass beads to the laminin; (c) placing the culture plate in an incubator at 37° C. for at least 12 hours in order to induce polymerization of laminin; and (d) removing excess PBS and allowing the culture plate to completely air dry.

### EXAMPLES

### Figures

FIG. 1 is a table showing the number of genes having at least a two-fold difference in expression levels (increase or decrease) from fibroblasts taken from 3 different patients cultured in tissue culture medium containing amniotic fluid growth medium and other media and various growth factors, as described in International Patent Publication No. WO 2009/151844, in order to drive propagation of multipotent stem cells, after passages 1, 2, and 3.

FIG. 2 is a Principle Component Analysis (PCA) plot showing the distribution of genes expressed from fibroblast-derived multipotent stem cells for 3 patients as the cells progress through adipose tissue differentiation, taken at day 0, 1, 3, 7, 10, 15, and 21.

FIG. 3 is a PCA plot showing the distribution of genes expressed from fibroblast-derived multipotent stem cells for 3 patients as the cells progress through hepatic tissue differentiation, taken at day 0, 1, 3, 6, 10, 12, 17, and 25.

FIG. 4 is a heat map and clustering diagram of the entire genome, comparing the gene expression of the undifferentiated fibroblast-derived multipotent stem cells from 3 patients against the gene expression of adult obese adipose tissue samples.

FIG. 5 is a heat map and clustering diagram of the entire genome, comparing the gene expression of the undifferentiated fibroblast-derived multipotent stem cells from 3 patients against the gene expression of adult lean adipose tissue samples.

FIG. 6 is a heat map and clustering diagram of the entire genome, comparing the gene expression of the undifferentiated fibroblast-derived multipotent stem cells from 3 patients against the gene expression of adult hepatic tissue samples.

FIG. 7 is a heat map and clustering diagram showing gene clusters that have at least a two-fold difference in expression levels from fibroblast-derived multipotent stem cells from 3 patients as the cells progress through adipose tissue differentiation, taken at day 7, 10, 15, and 21.

FIG. 8 is a heat map and clustering diagram showing gene clusters that have at least a two-fold difference in expression levels from fibroblast-derived multipotent stem cells from 3 patients as the cells progress through hepatic tissue differentiation, taken at day 6, 10, 12, 17, and 25.

FIG. 9 is a heat map and clustering diagram of an individual 37 year old patient (Sample 970) showing the genes that have at least a two-fold difference in expression levels from the patient's fibroblasts samples (Control) as the cells differentiate into adipose tissue, taken at 24 hours, 3 days, 7 days, 10 days, 15 days, and 21 days after culture in media that promotes differentiation into adipose tissue.

FIG. 10 is a heat map and clustering diagram of an individual 3 day old patient (Sample 1650) showing the genes that have at least a two-fold difference in expression levels from the patient's fibroblasts samples (Control) as the cells differentiate into adipose tissue, taken at 24 hours, 3 days, 7 days, 10 days, 15 days, and 21 days after culture in media that promotes differentiation into adipose tissue.

FIG. 11 is a heat map and clustering diagram of an individual 96 year old patient (Sample 731) showing the genes that have at least a two-fold difference in expression levels from the patient's fibroblasts samples (Control) as the cells differentiate into adipose tissue, taken at 24 hours, 3 days, 7 days, 10 days, 15 days, and 21 days after culture in media that promotes differentiation into adipose tissue

FIG. 12 is a summary of the data from FIGS. 9-11.

FIG. 13 is a heat map and clustering diagram of the entire genome, comparing the gene expression of the differentiated adipose tissue from the 3 patients (after 21 days in media that promotes differentiation into adipose tissue) against the gene expression of adult lean adipose tissue samples.

FIG. 14 is a heat map and clustering diagram of the entire genome, comparing the gene expression of the differentiated adipose tissue from the 3 patients (after 21 days in media that promotes differentiation into adipose tissue) against the gene expression of adult obese adipose tissue samples.

FIG. 15 is a heat map and clustering diagram of the entire genome, comparing the gene expression of adult obese adipose tissue samples against the gene expression of adult lean adipose tissue samples.

FIG. 16 is a heat map and clustering diagram of the entire genome, comparing the gene expression of the differentiated hepatic tissue from the 3 patients (after 25 days in media that promotes differentiation into hepatic tissue) against the gene expression of adult liver tissue samples.

FIG. 17 is a collection of data showing a heat map and clustering diagram of the entire genome, comparing the gene expression of fibroblast-derived multipotent stem cells, differentiated adipose tissue from the 3 patients (after 21 days in media that promotes differentiation into adipose tissue), and adult lean adipose tissue samples, and adult obese adipose tissue samples.

FIG. 18 is a collection of data showing a heat map and clustering diagram of the entire genome, comparing the gene expression of fibroblast-derived multipotent stem cells, differentiated hepatic tissue from the 3 patients (after 25 days in media that promotes differentiation into hepatic tissue), and adult hepatic tissue samples.

FIG. 19 is a PCA plot showing the distribution of genes expressed from fibroblast-derived multipotent stem cells for 3 patients as the cells progress through muscle tissue differentiation, taken at day 0, 1.5, 4.5, 7.5, 10.5, 13.5, 16.5, and 19.5.

FIG. 20 is a PCA plot showing the distribution of genes expressed from fibroblast-derived multipotent stem cells for 3 patients as the cells progress through nerve tissue differentiation, taken at day 0, 0.5, 1, 2, 4, 6, and 8.

FIG. 21 is a heat map and clustering diagram showing gene clusters that have at least a two-fold difference in expression levels from fibroblast-derived multipotent stem cells from 3 patients as the cells progress through muscle tissue differentiation, taken at day 1.5, 4.5, 7.5, 10.5, 13.5, 16.5, and 19.5.

FIG. 22 is a PCA plot showing the distribution of genes expressed from skin fibroblasts from 3 patients when cultured in: (a) conventional media (Eagles-based MEM); and (b) media that propagates multipotent stem cells (α-MEM media plus supplements).

FIG. 23, like FIG. 8, is a heat map and clustering diagram showing gene clusters that have at least a two-fold difference in expression levels from fibroblast-derived multipotent stem cells from 3 patients as the cells progress through hepatic tissue differentiation, however, taken at day 7, 10, 15, and 21.

FIG. 24 is a heat map and clustering diagram of an individual 37 year old patient (Sample 970) showing the genes that have at least a two-fold difference in expression levels from the patient's fibroblasts samples (Control) as the cells differentiate into hepatic tissue, taken at 24 hours, 3 days, 6 days, 10 days, 12 days, 17 days, and 25 days after culture in media that promotes differentiation into hepatic tissue.

FIG. 25 is a heat map and clustering diagram of an individual 3 day old patient (Sample 1650) showing the genes that have at least a two-fold difference in expression levels from the patient's fibroblasts samples (Control) as the cells differentiate into hepatic tissue, taken at 24 hours, 3 days, 6 days, 10 days, 12 days, 17 days, and 25 days after culture in media that promotes differentiation into hepatic tissue.

FIG. 26 is a heat map and clustering diagram of an individual 96 year old patient (Sample 731) showing the genes that have at least a two-fold difference in expression levels from the patient's fibroblasts samples (Control) as the cells differentiate into hepatic tissue, taken at 24 hours, 3 days, 6 days, 10 days, 12 days, 17 days, and 25 days after culture in media that promotes differentiation into hepatic tissue.

FIG. 27 is a summary of the data from FIGS. 24-26.

FIG. 28, like FIG. 8 and FIG. 23. shows a heat map and clustering diagram showing gene clusters that have at least a two-fold difference in expression levels from fibroblast-derived multipotent stem cells from 3 patients as the cells progress through hepatic tissue differentiation, however, taken at day 1, 3, 67, 10, 15, and 21.

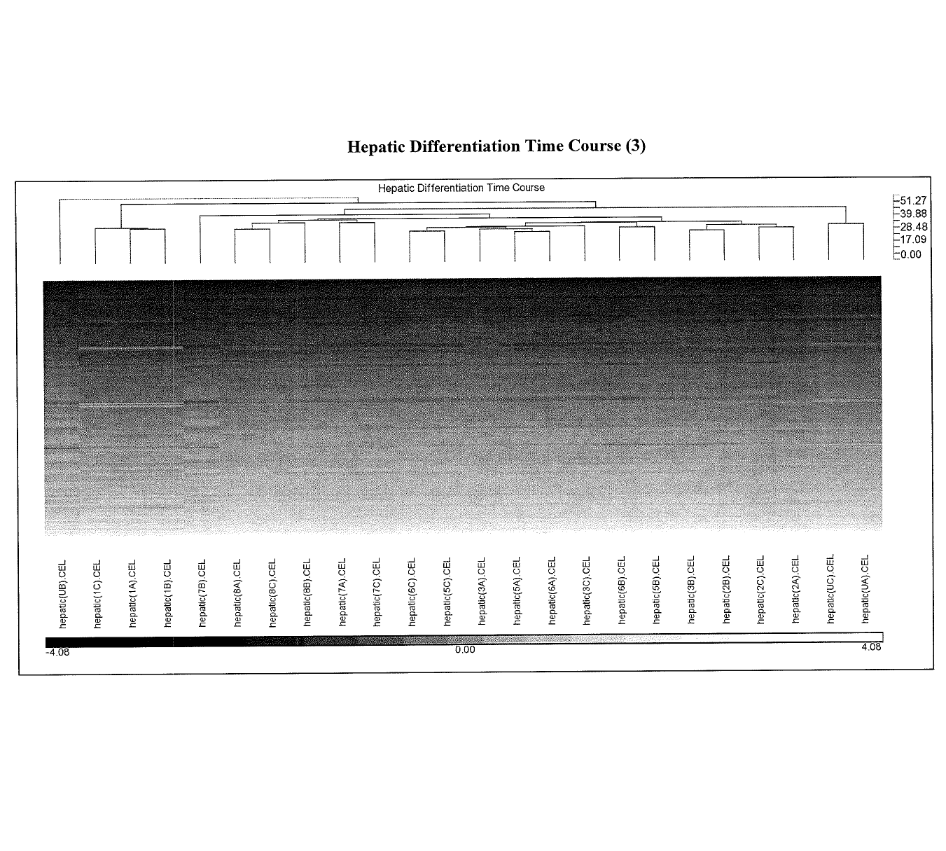

FIG. 29 is a PCA plot showing the distribution of genes expressed from fibroblast-derived multipotent stem cells for 3 patients as the cells progress through nerve tissue differentiation, taken at day 0, 0.5, 1, 2, 4, 6, and 8.

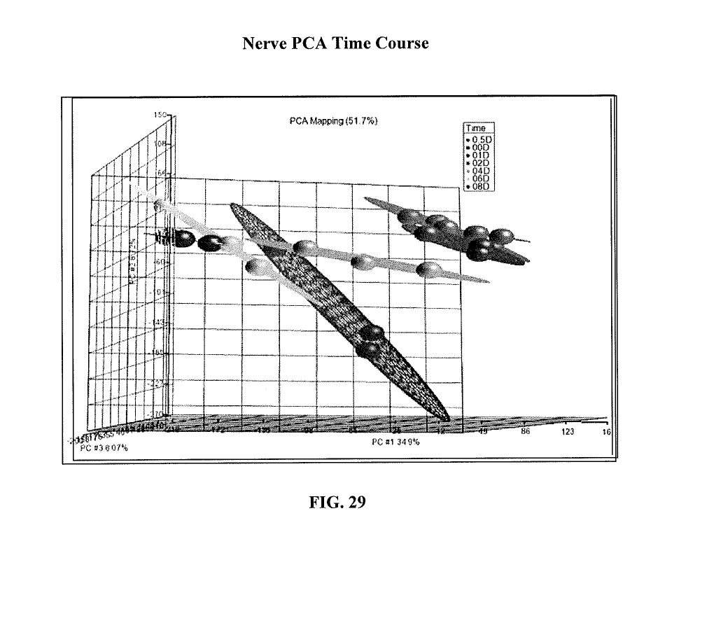

FIG. 30, a summary of FIGS. 34-36, is a heat map and clustering diagram of 3 patients showing the genes that have at least a two-fold difference in expression levels from the patient's fibroblasts samples (Control) as the cells differentiate into nerve tissue, taken at 12 hours, 24 hours, 2 days, 4 days, 6 days, and 8 days after culture in media that promotes differentiation into nerve tissue.

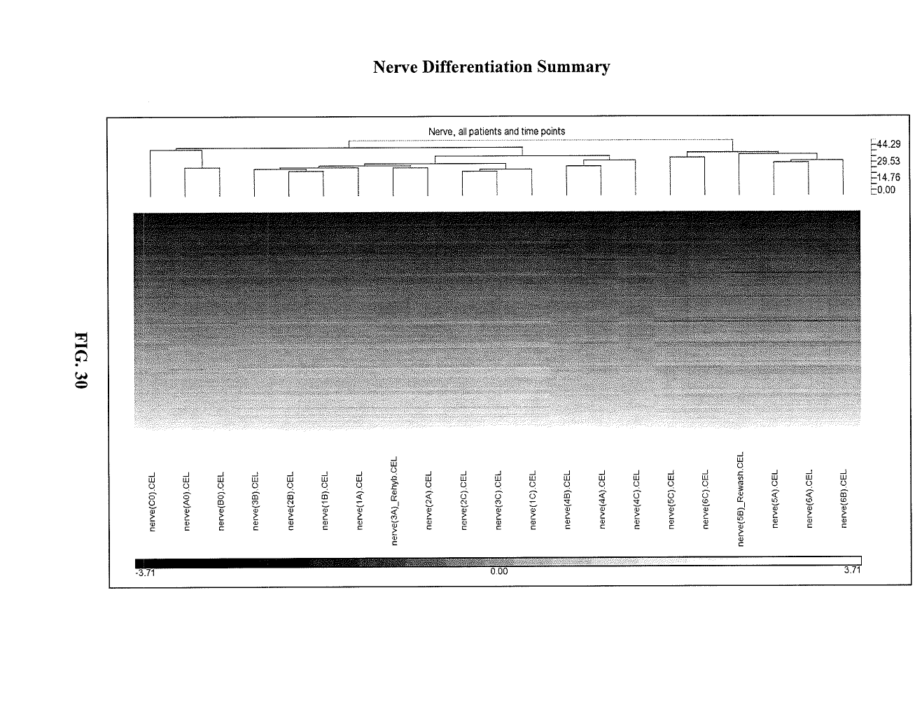

FIG. 31 is a heat map and clustering diagram of an individual 96 year old patient (Sample 731) showing the genes that have at least a two-fold difference in expression levels from the patient's fibroblasts samples (Control) as the cells differentiate into muscle tissue, taken at 1.5 days, 4.5 days, 7.5 days, 10.5 days, 13.5 days, and 16.5 days after culture in media that promotes differentiation into muscle tissue.

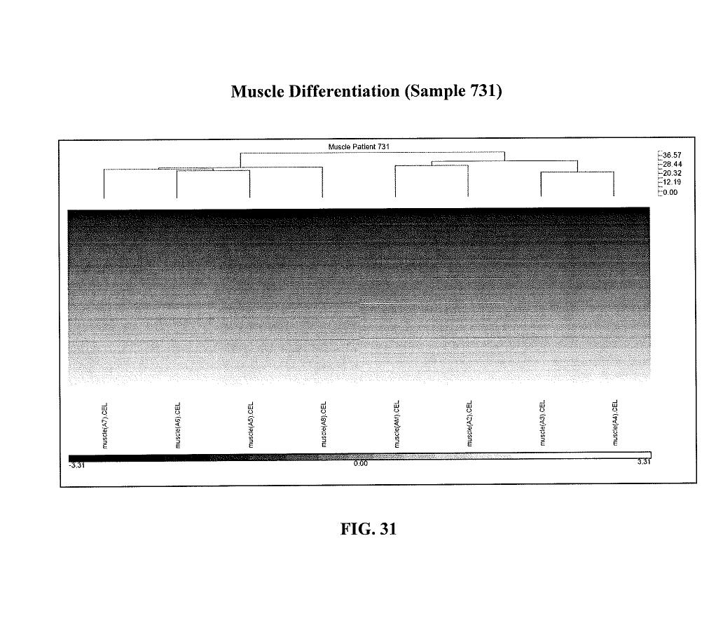

FIG. 32 is a heat map and clustering diagram of an individual 37 year old patient (Sample 970) showing the genes that have at least a two-fold difference in expression levels from the patient's fibroblasts samples (Control) as the cells differentiate into muscle tissue, taken at 1.5 days, 4.5 days, 7.5 days, 10.5 days, 13.5 days, and 16.5 days after culture in media that promotes differentiation into muscle tissue.

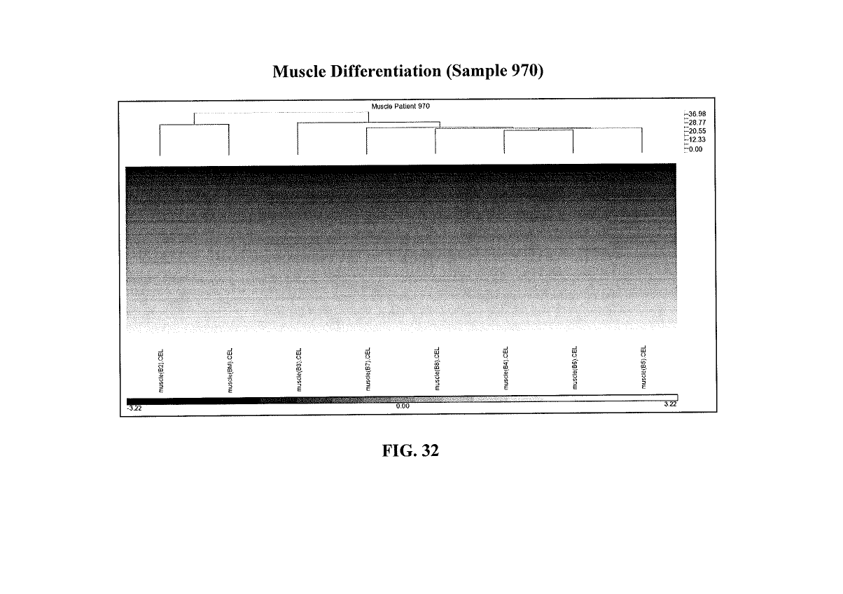

FIG. 33 is a heat map and clustering diagram of an individual 3 day old patient (Sample 1650) showing the genes that have at least a two-fold difference in expression levels from the patient's fibroblasts samples (Control) as the cells differentiate into muscle tissue, taken at 1.5 days, 4.5 days, 7.5 days, 10.5 days, 13.5 days, and 16.5 days after culture in media that promotes differentiation into muscle tissue.

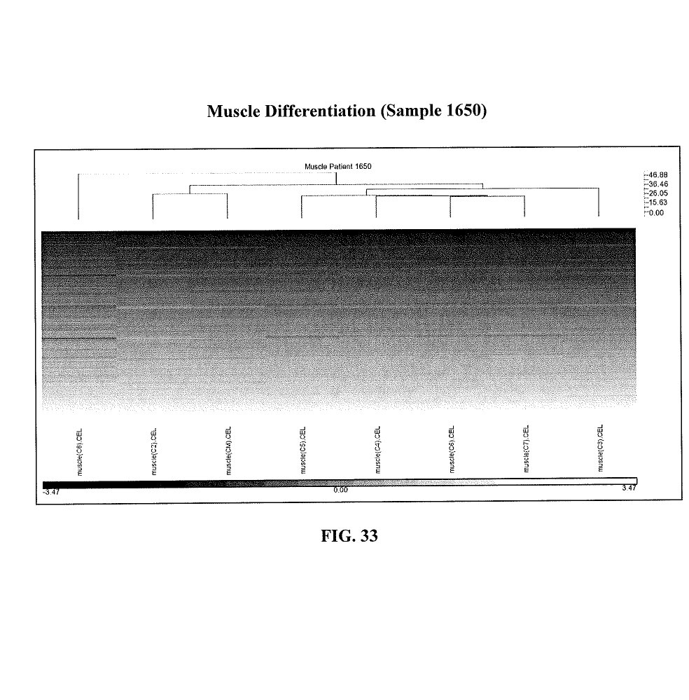

FIG. 34 is a heat map and clustering diagram of an individual 96 year old patient (Sample 731) showing the genes that have at least a two-fold difference in expression levels from the patient's fibroblasts samples (Control) as the cells differentiate into nerve tissue, taken at 0.5, 1, 2, 4, 6, and 8 days after culture in media that promotes differentiation into nerve tissue.

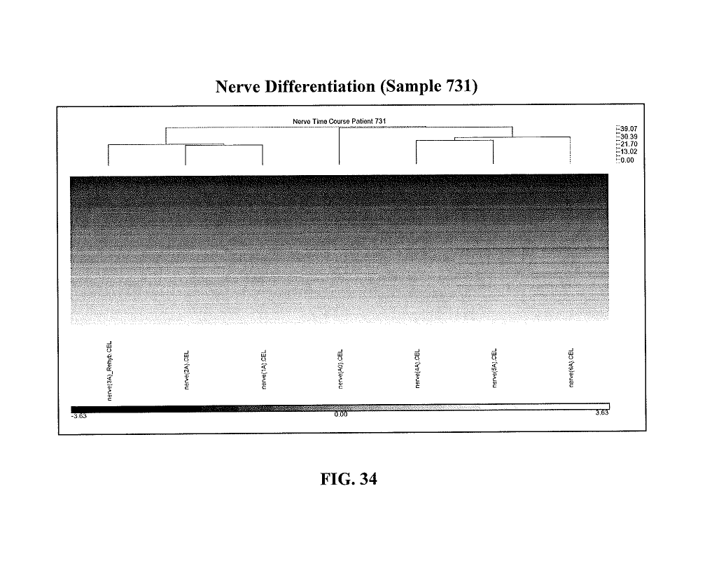

FIG. 35 is a heat map and clustering diagram of an individual 37 year old patient (Sample 970) showing the genes that have at least a two-fold difference in expression levels from the patient's fibroblasts samples (Control) as the cells differentiate into nerve tissue, taken at 0.5, 1, 2, 4, 6, and 8 days after culture in media that promotes differentiation into nerve tissue.

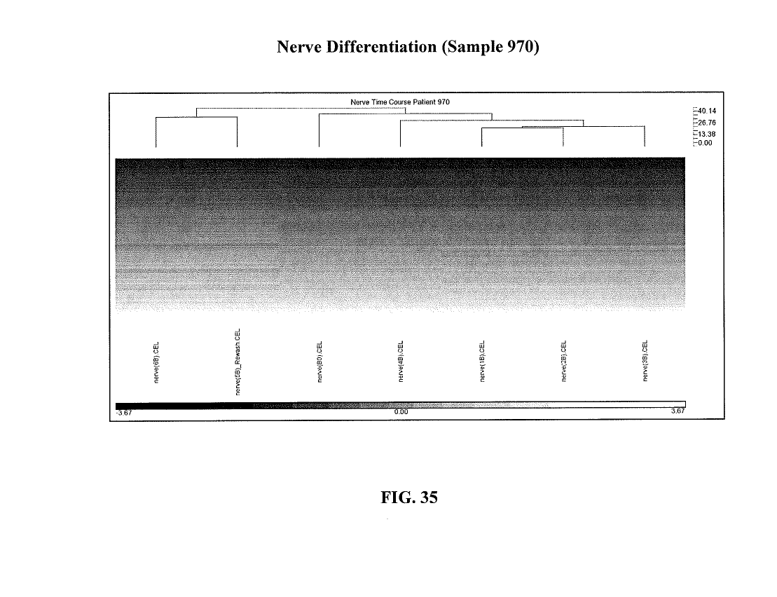

FIG. 36 is a heat map and clustering diagram of an individual 3 day old patient (Sample 1650) showing the genes that have at least a two-fold difference in expression levels from the patient's fibroblasts samples (Control) as the cells differentiate into nerve tissue, taken at 0.5, 1, 2, 4, 6, and 8 days after culture in media that promotes differentiation into nerve tissue.

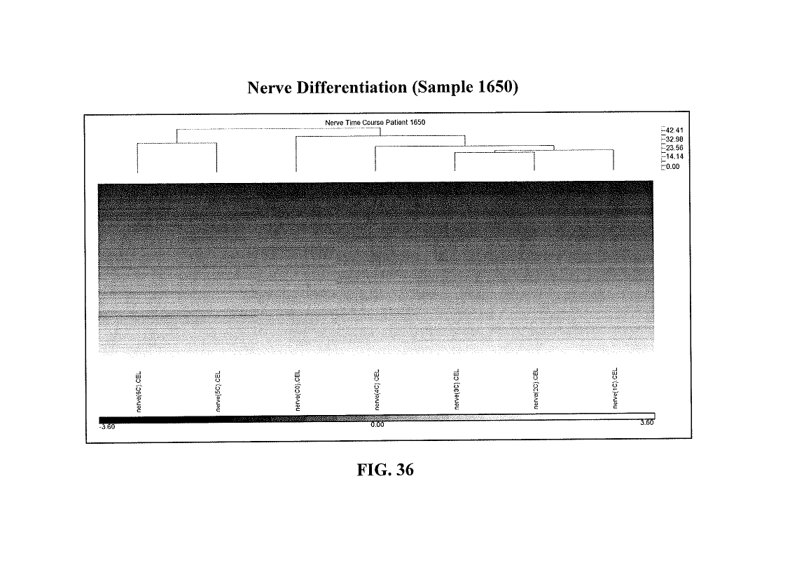

### EXAMPLE 1

Fibroblast were obtained from 3 different patients ranging in age from 3 days old (Sample 1650); 37 years old (Sample 970); and 96 years old (Sample 731). The fibroblasts were cultured in medium comprising amniotic growth fluid media (AFM) (as described in DeCoppi et al., comprising α-MEM (Invitrogen), 15% ES-FBS (Invitrogen), 1% L-Glutamine, and 1% Pen/Strep, supplemented with 18% CHANG MEDIUM® B (Irvine Scientific) and 2% CHANG MEDIUM® C (Irvine Scientific)), so as to propagate multipotent stem cells, as is described in International Patent Publication No. WO 2009/151844. The AFM comprises α-MEM media plus supplements.

After passages 1, 2, and 3, cells were harvested and subject to gene expression profiling using the Affymetrix GENECHIP® (Affymetrix GENECHIP® microarray technology) Human Gene 1.0 ST Array, as described in International Patent Publication No. WO 2009/151844.

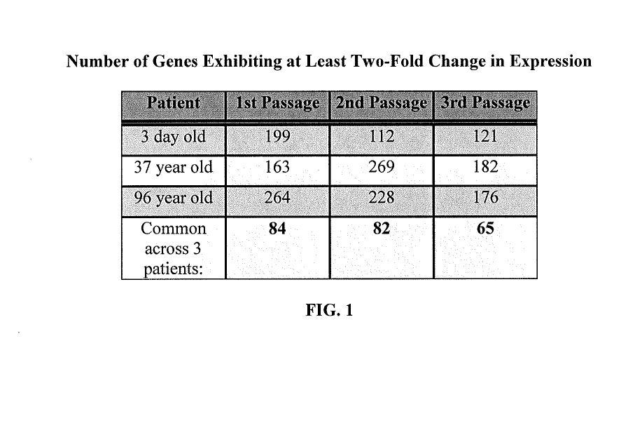

FIG. 1 is a table that shows the number of genes that exhibit at least a two-fold difference in gene expression (increase or decrease) of fibroblast cells cultured in tissue culture medium containing amniotic fluid growth medium and other media and various growth factors, as described in International Patent Publication No. WO 2009/151844, in order to drive propagation of multipotent stem cells, after passages 1, 2, and 3, as compared against the gene expression of the resting fibroblasts in traditional MEM media as described in International Patent Publication No. WO 2009/151844. Additionally, the table shows the number of genes exhibit at least a two-fold difference in expression levels (increase or decrease) that are common across all 3 patients. These studies demonstrate that these fibroblast cells obtained from 3 very different individuals are upregulating and downregulating a fair number of the same genes when subject to the same media conditions. This suggests that the process of obtaining multipotency may involve a genetic expression profile shared by all humans, regardless of age.

### EXAMPLE 2

The fibroblast-derived multipotent stem cells from the 3 patients in Example 1 were subject to differentiation media conditions suitable to promote lineage-specific differentiation as described in International Patent Publication No. WO 2009/151844.

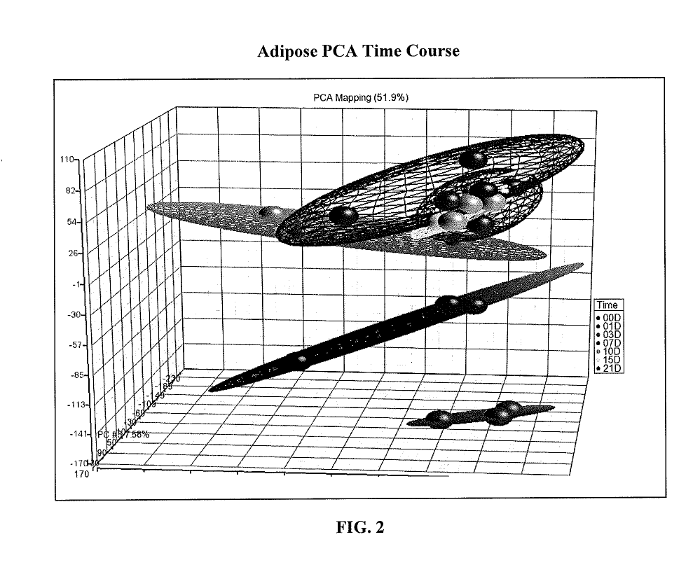

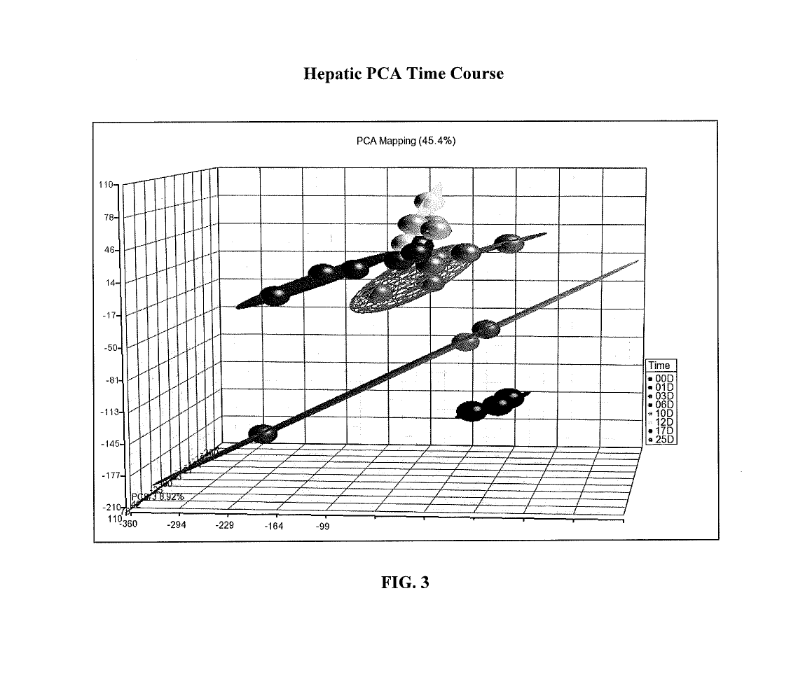

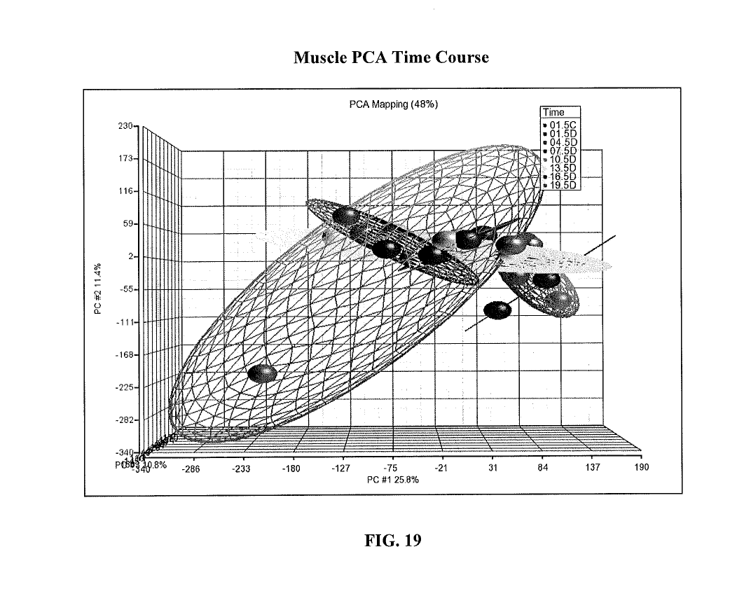

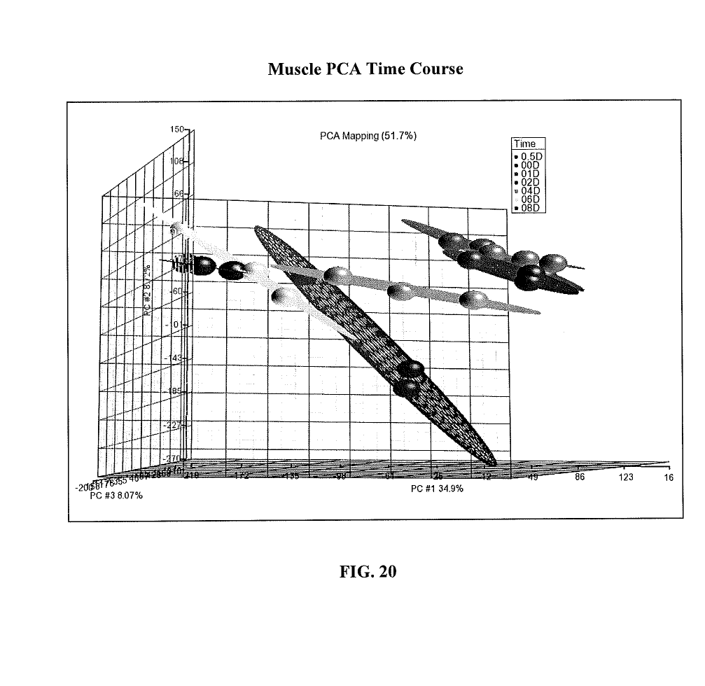

FIG. 2 depicts a Principle Component Analysis (PCA) plot showing the distribution of genes expressed from fibroblast-derived multipotent stem cells for the 3 patients as the cells progress through adipose tissue differentiation, taken at day 0, 1, 3, 7, 10, 15, and 21 of culture. Each of the 3 patients is represented by a sphere. Similarly, FIG. 3 depicts a PCA plot showing the distribution of genes expressed from fibroblast-derived multipotent stem cells for the 3 patients as the cells progress through hepatic tissue differentiation, taken at day 0, 1, 3, 6, 10, 12, 17, and 25. FIG. 19 depicts a PCA plot showing the distribution of genes expressed from fibroblast-derived multipotent stem cells for the 3 patients as the cells progress through muscle tissue differentiation, taken at day 0, 1.5, 4.5., 7.5, 10.5, 13.5, 16.5, and 19.5. FIG. 20 depicts a PCA plot showing the distribution of genes expressed from fibroblast-derived multipotent stem cells for the 3 patients as the cells progress through nerve tissue differentiation, taken at day 0, 0.5, 1, 2, 4, 6, and 8. The clustering of the patient data points, in FIGS. 2, 3, 19, and 20, at each time point indicates that the cells for patient are undergoing many of the same genetic expression changes when undergoing the same lineage-specific differentiation process.

### EXAMPLE 3

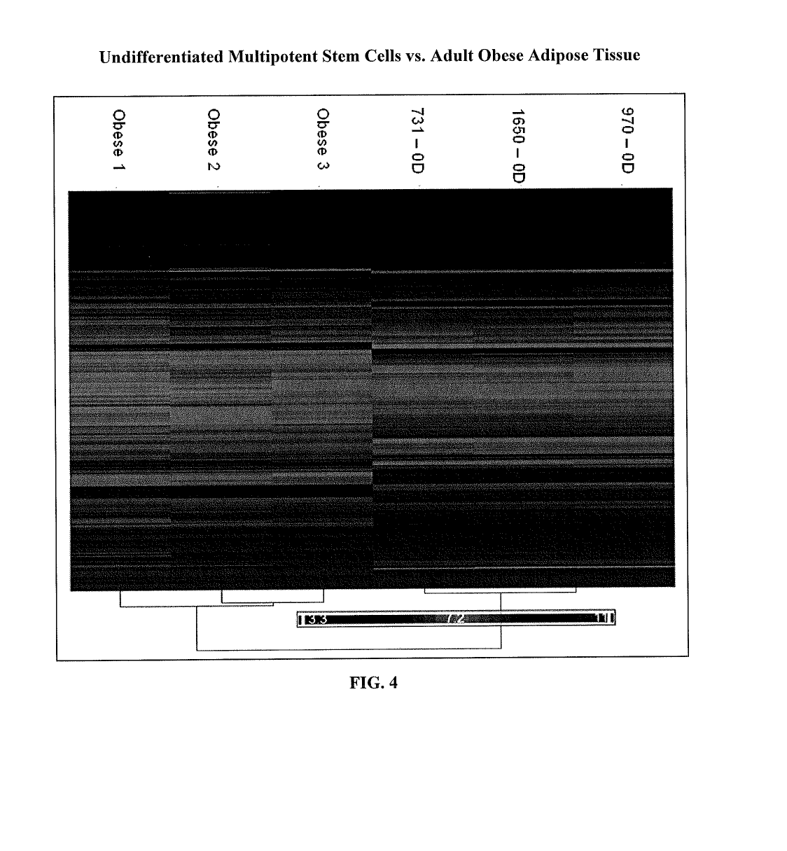

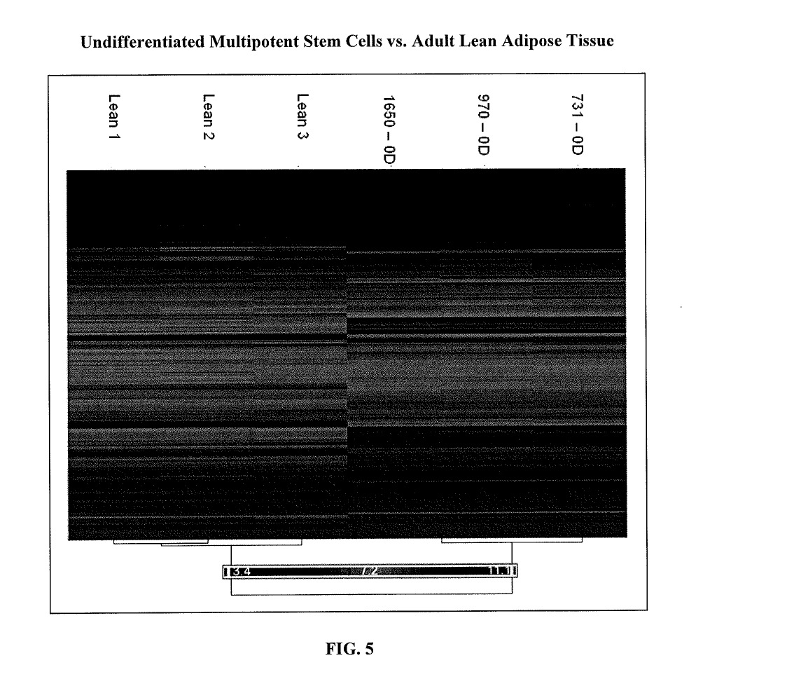

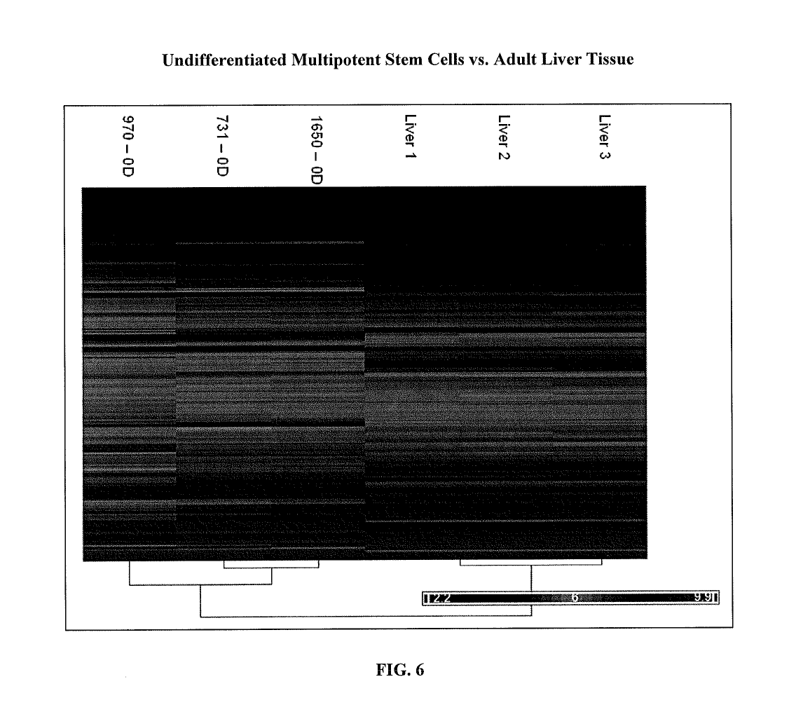

Undifferentiated fibroblast-derived multipotent stem cells from the 3 patient samples and adult obese adipose tissue samples, adult lean adipose tissue samples, and adult liver tissue samples were subject to microarray analysis as described in International Patent Publication No. WO 2009, which protocols, methods, and materials are incorporated herein by reference. The data from the adult obese adipose, adult lean adipose, and adult liver tissue samples, subject to the same microarray analysis, were obtained from the Gene Expression Omnibus at the National Center for Biotechnology Information, NIH. FIGS. 4-6 show heat maps and clustering diagrams of the whole genome showing gene expression profiles of the undifferentiated fibroblast-derived multipotent stem cells against obese adipose tissue (FIG. 4), lean adipose tissue (FIG. 5), and liver tissue (FIG. 6). Differential expression is observed by intensity of color (black or white) for the expression level of that corresponding gene. The data from FIGS. 4-6 show similar gene expression profiles as between the 3 different patients and similar gene expression profiles as between the individual adult tissues. However, the gene expression profiles are dramatically different as between the undifferentiated fibroblast-derived multipotent stem cells and any of the adult tissue samples.

### EXAMPLE 4

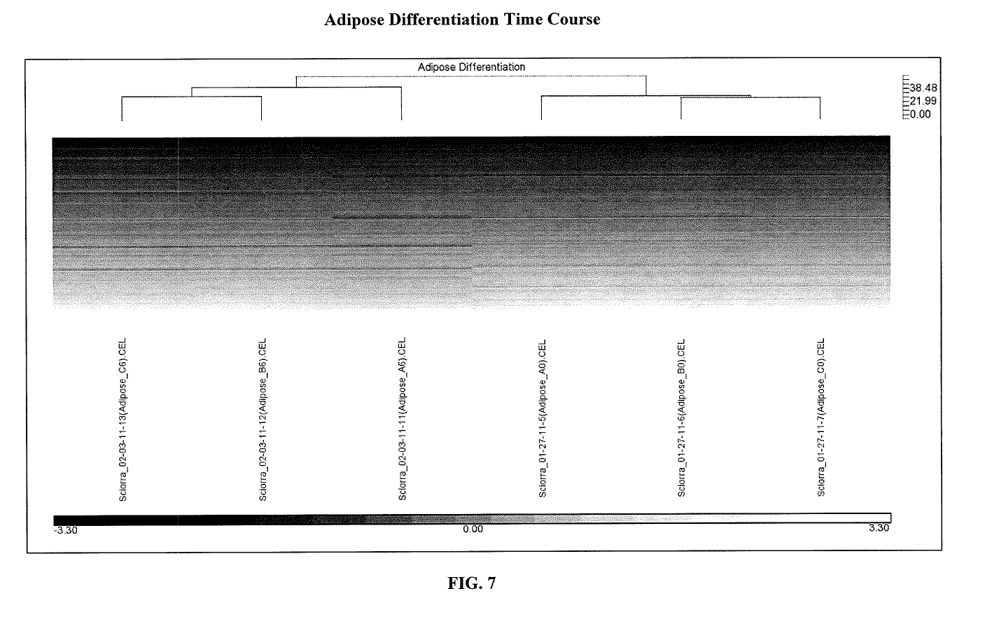

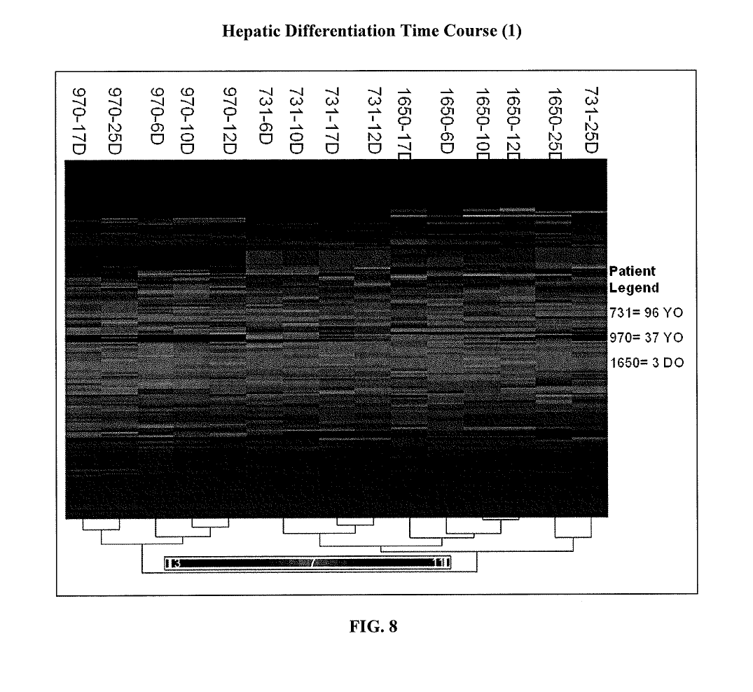

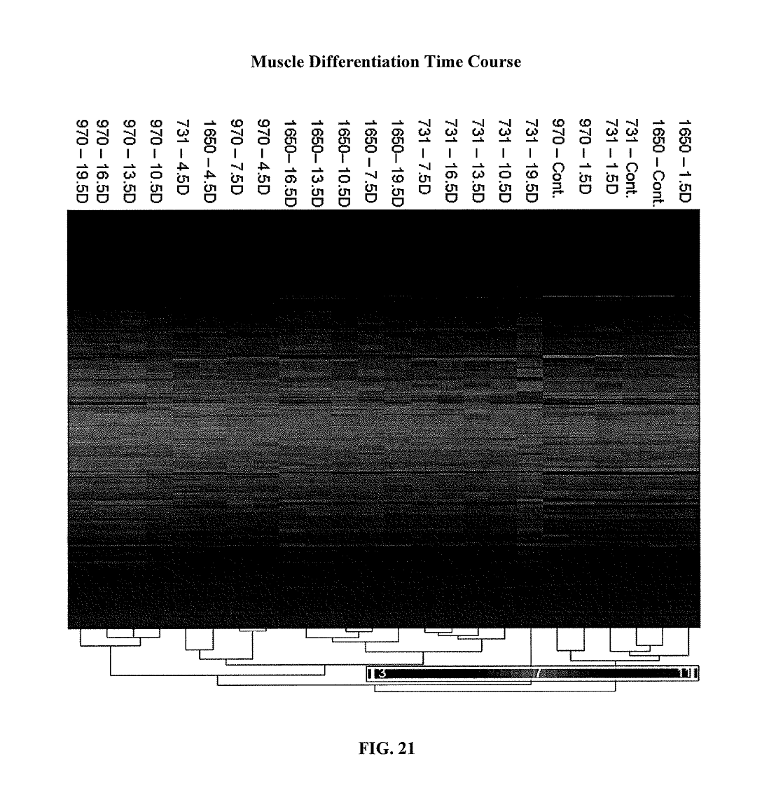

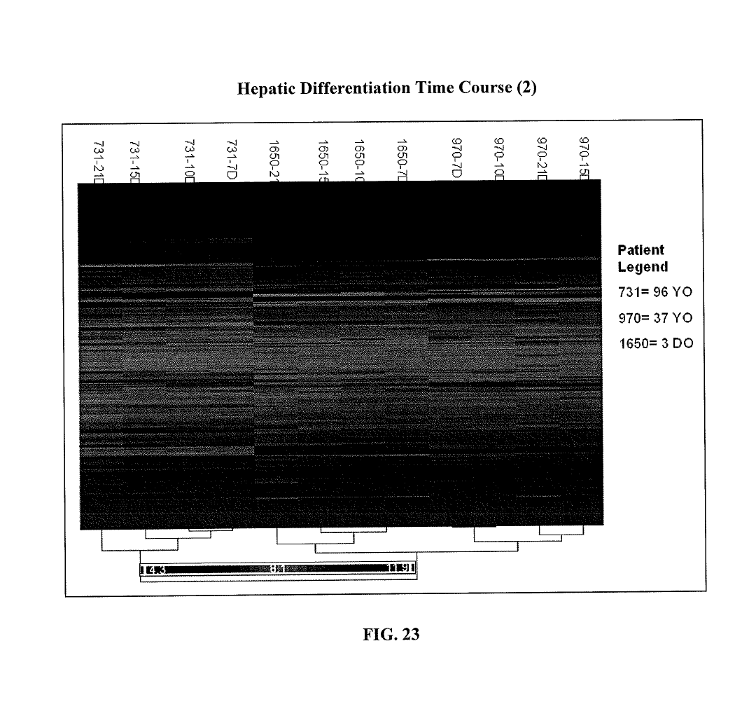

The fibroblast-derived multipotent stem cells from the 3 patients were subject to differentiation media conditions suitable to promote lineage-specific differentiation as described in International Patent Publication No. WO 2009/151844, which protocols, methods, and materials are incorporated herein by reference. At various time points during the differentiation process, cells were collected and were subject to a microarray analysis as described in International Patent Publication No. WO 2009/151844. For adipose cell differentiation, cells were harvested at day 7, 10, 15, and 21; for hepatic differentiation, cells were harvested at day 6, 10, 12, and 25; and for muscle differentiation, cells were harvested at day 1.5, 4.5, 7.5, 10.5, 13.5, 16.5, and 19.5. FIG. 7 shows a heat map and clustering diagram showing the expression profiles of genes exhibiting at least a two-fold change in expression for the fibroblast-derived multipotent stem cells from the 3 patients undergoing adipose cell differentiation. FIGS. 8 and 23 shows a heat map and clustering diagram showing the expression profiles of clusters of genes exhibiting at least a two-fold change in expression (increase or decrease) for the fibroblast-derived multipotent stem cells from the 3 patients undergoing hepatic cell differentiation. FIG. 21 shows a heat map and clustering diagram showing the expression profiles of clusters of genes exhibiting at least a two-fold change in expression (increase or decrease) for the fibroblast-derived multipotent stem cells from the 3 patients undergoing muscle cell differentiation. The data in FIGS. 7, 8, 21, and 23 show that the patient samples exhibit increasingly similar gene expression profiles toward the end of the differentiation cycle. Indeed, the gene expression profiles toward the end of the differentiation cycle resemble the gene expression profiles of the adult tissues samples from Example 3 (FIGS. 4-6).

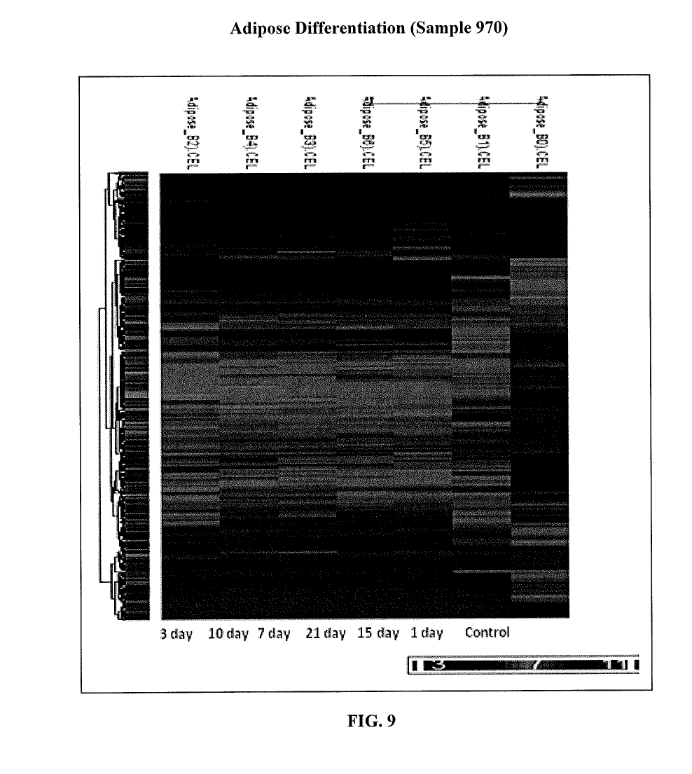

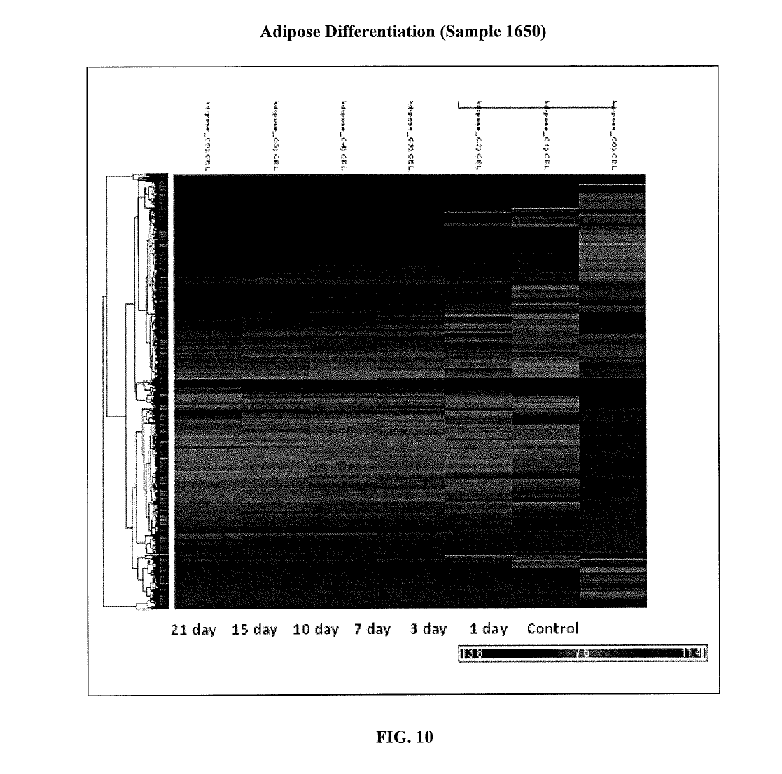

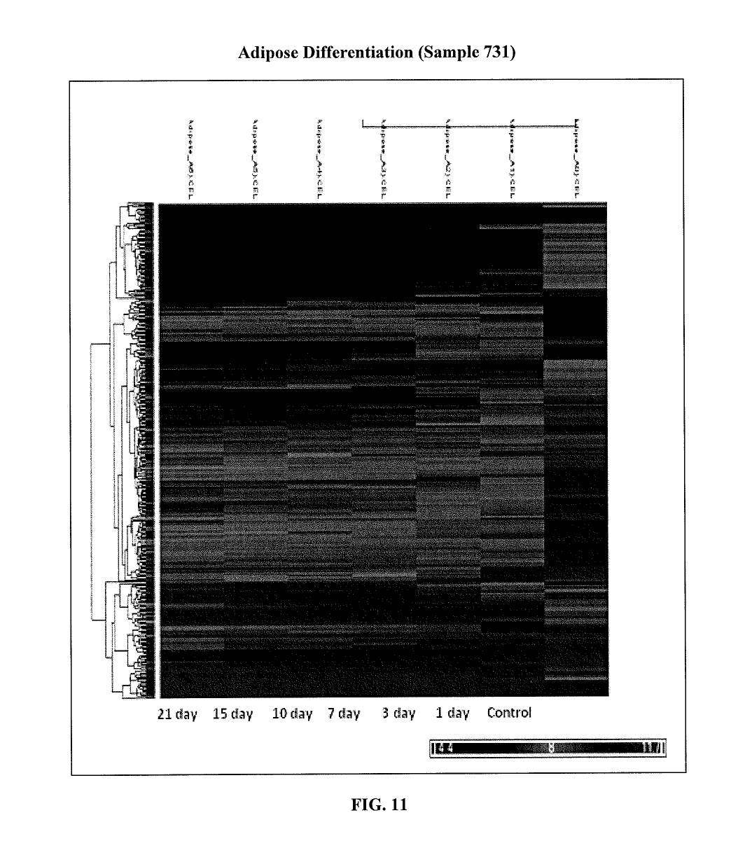

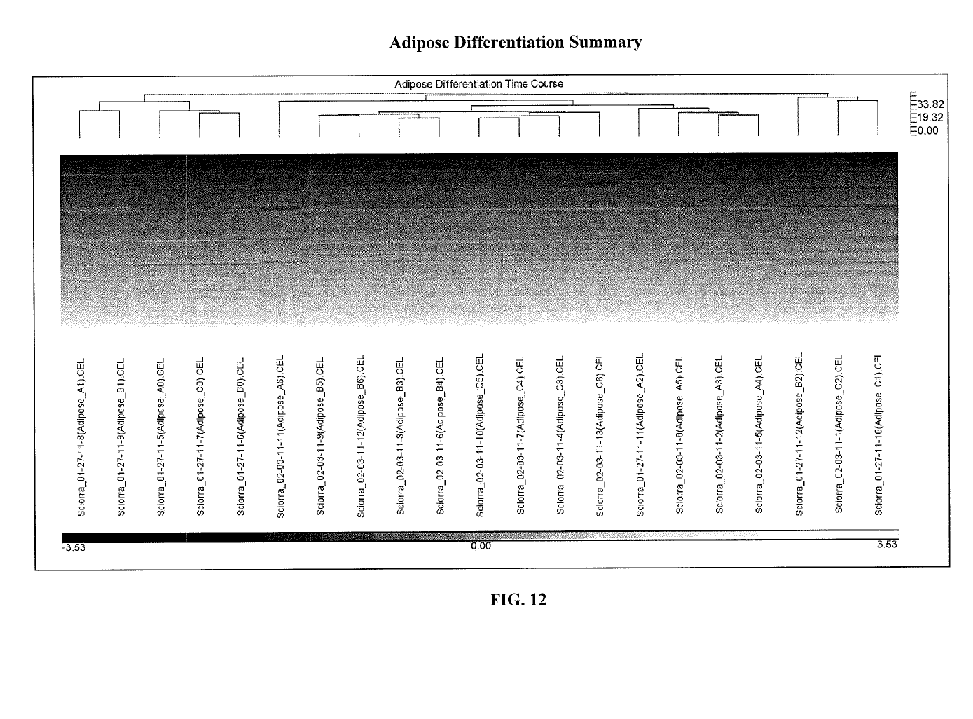

These data were also analyzed on an individual patient basis. FIGS. 9-11 contain heat maps and cluster diagrams showing the expression profiles of genes exhibiting at least a two-fold change in expression (increase or decrease) for the fibroblast-derived multipotent stem cells from the 3 patients undergoing adipose cell differentiation at 1, 3, 7, 10, 15, and 21 days for Sample 970 (37 year old) (FIG. 9), Sample 1650 (3 day old) (FIG. 10), and Sample 731 (96 year old) (FIG. 11). FIG. 12 is a summary of the data from FIGS. 9-11. FIG. 12 supports the data of FIGS. 7, 8, 21, and 23, showing that the patient samples exhibit increasingly similar gene expression profiles toward the end of the differentiation cycle.

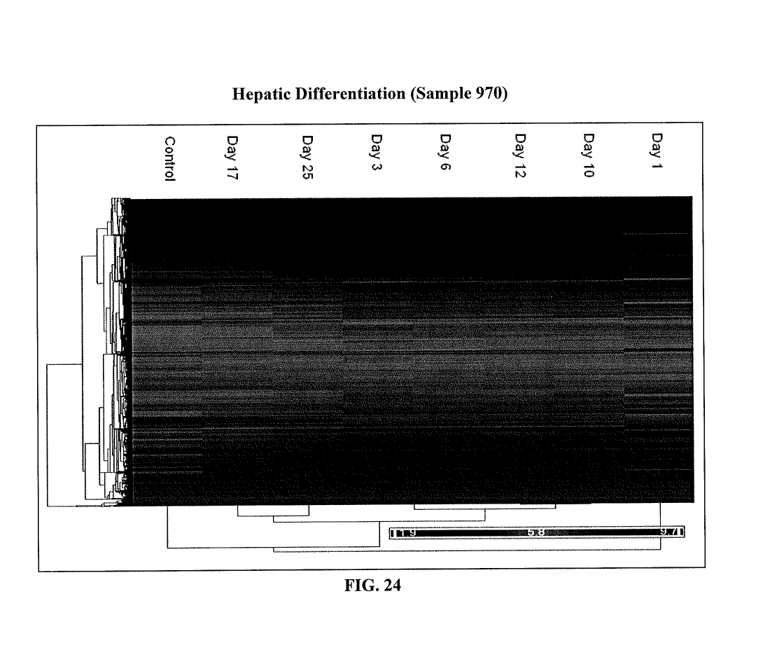

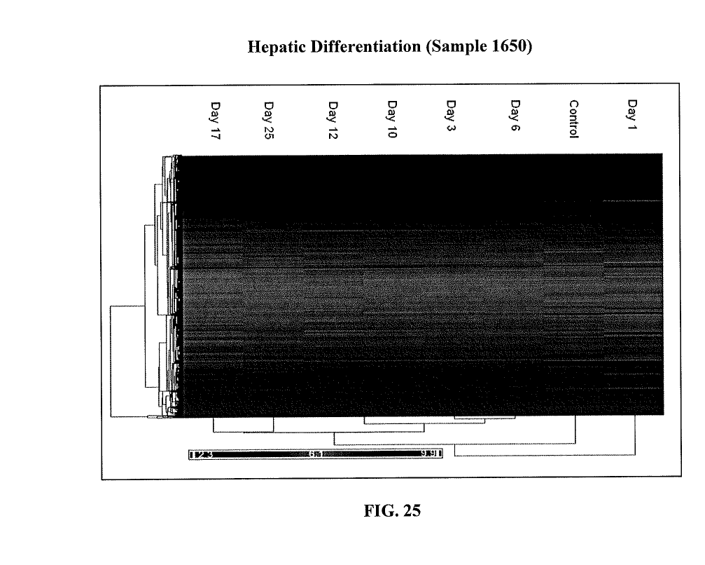

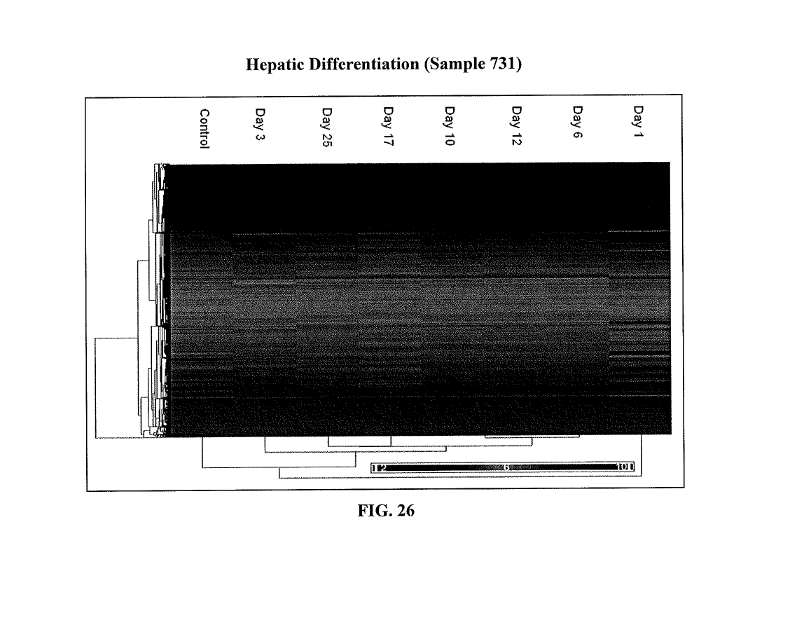

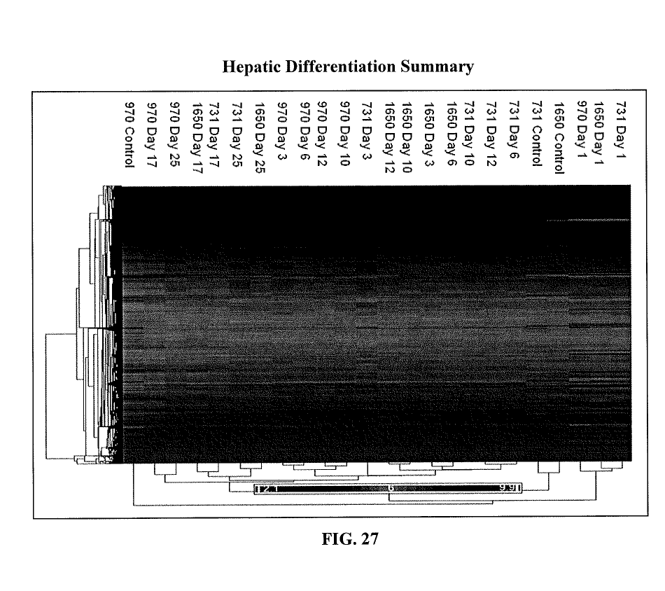

Similarly, FIGS. 24-26 contain heat maps and cluster diagrams showing the expression profiles of genes exhibiting at least a two-fold change in expression (increase or decrease) for the fibroblast-derived multipotent stem cells from the 3 patients undergoing hepatic cell differentiation at 1, 3, 6, 10, 12, 17, and 25 days for Sample 970 (37 year old) (FIG. 24), Sample 1650 (3 day old) (FIG. 25), and Sample 731 (96 year old) (FIG. 26). FIG. 27 is a summary of the data from FIGS. 24-26. FIG. 27 supports the data of showing that the patient samples exhibit increasingly similar gene expression profiles toward the end of the differentiation cycle.

### EXAMPLE 5

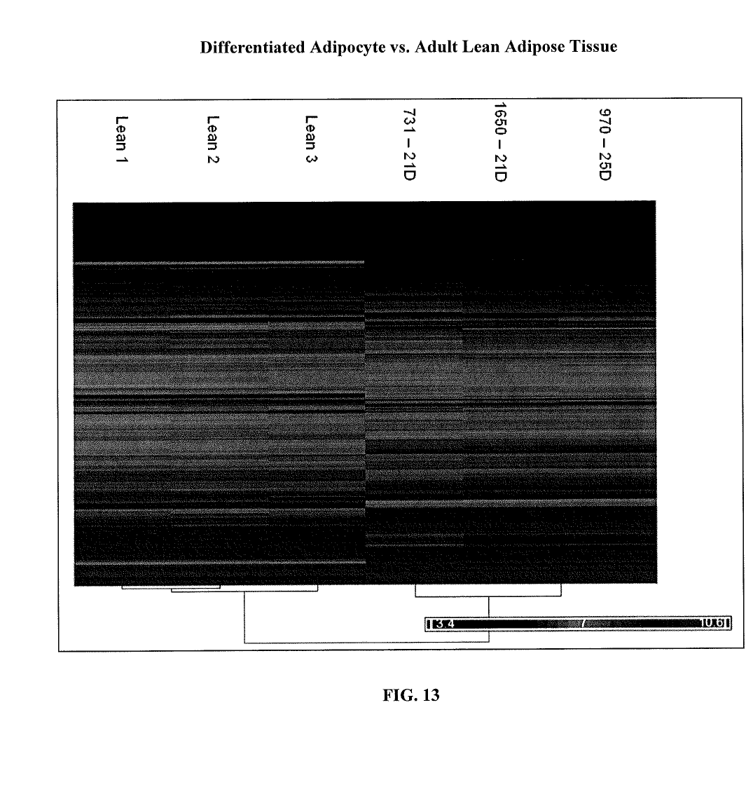

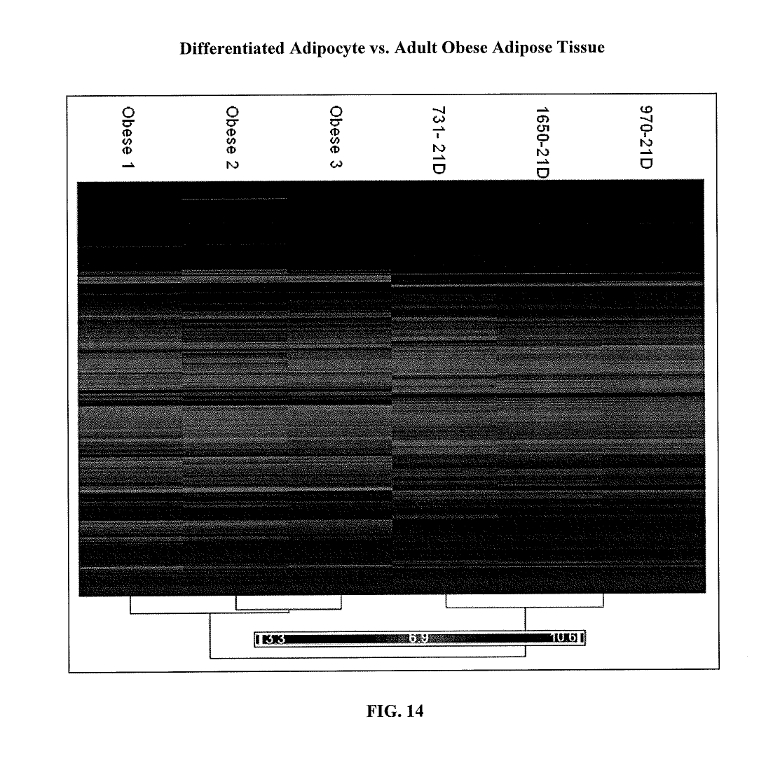

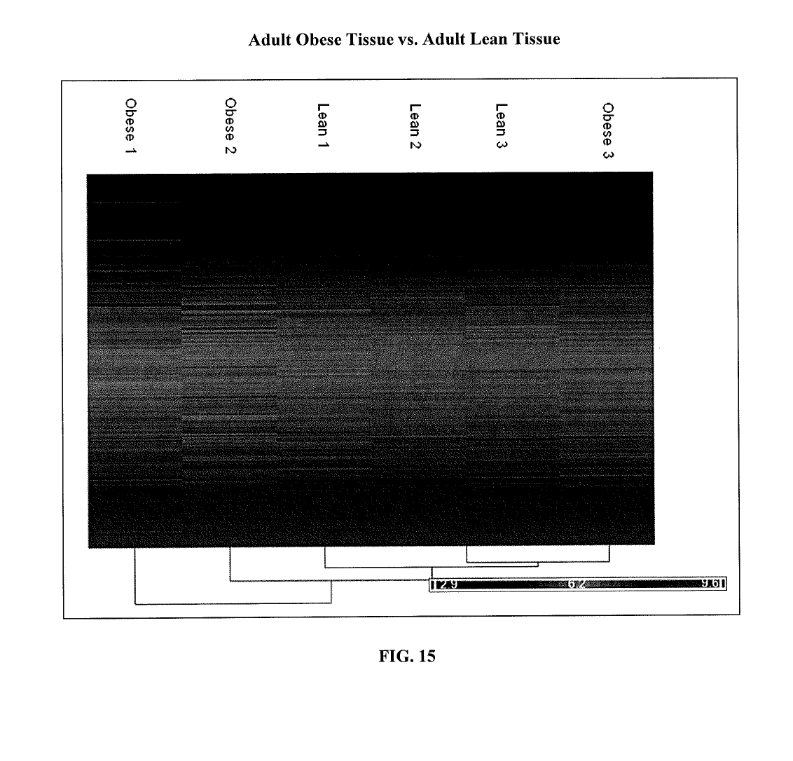

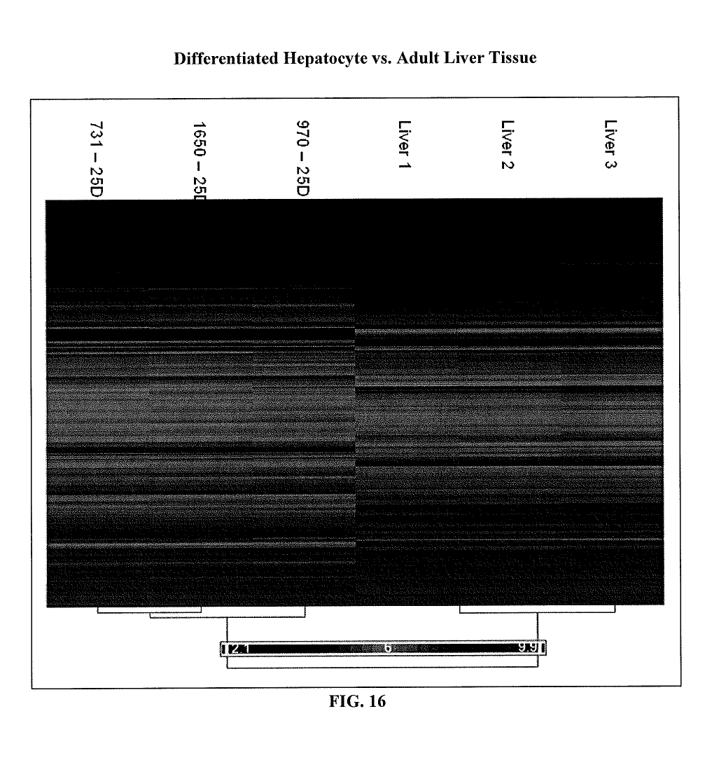

Studies were conducted to determine if the lineage-specific differentiated cells, which were derived from fibroblasts, show similar gene expression profiles as those actual adult cells of the same corresponding lineage. To that end, fibroblast-derived multipotent stem cells from the 3 patient samples were subject to differentiation under conditions of adipose cell differentiation (for 21 days) or hepatic cell differentiation (for 25 days). These differentiated fibroblast-derived adipose cells were subject to microarray analysis along with samples of adult lean adipose tissue (FIG. 13) and adult obese adipose tissue (FIG. 14). Similarly, these differentiated fibroblast-derived hepatic cells were subject to microarray analysis along with samples of adult liver tissue (FIG. 16). The data from the adult obese adipose, adult lean adipose, and adult liver tissue samples, subject to the same microarray analysis, were obtained from the Gene Expression Omnibus at the National Center for Biotechnology Information, NIH. FIG. 13 and FIG. 14 show a heat map and cluster diagram showing the expression profiles of the whole genome for differentiated fibroblast-derived adipose cells against adult lean adipose tissue and adult obese adipose tissue, respectively. FIG. 15 shows a heat map and cluster diagram showing the expression profiles of the whole genome for adult lean adipose tissue and adult obese adipose tissue. FIG. 16 shows a heat map and cluster diagram showing the expression profiles of the whole genome for differentiated fibroblast-derived hepatic cells against adult liver tissue.

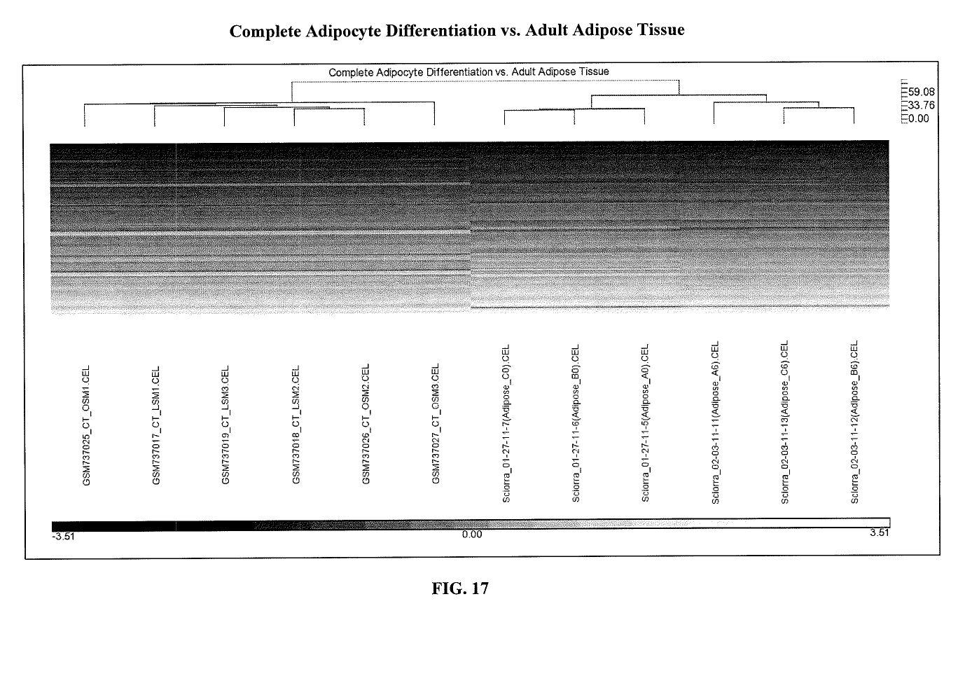

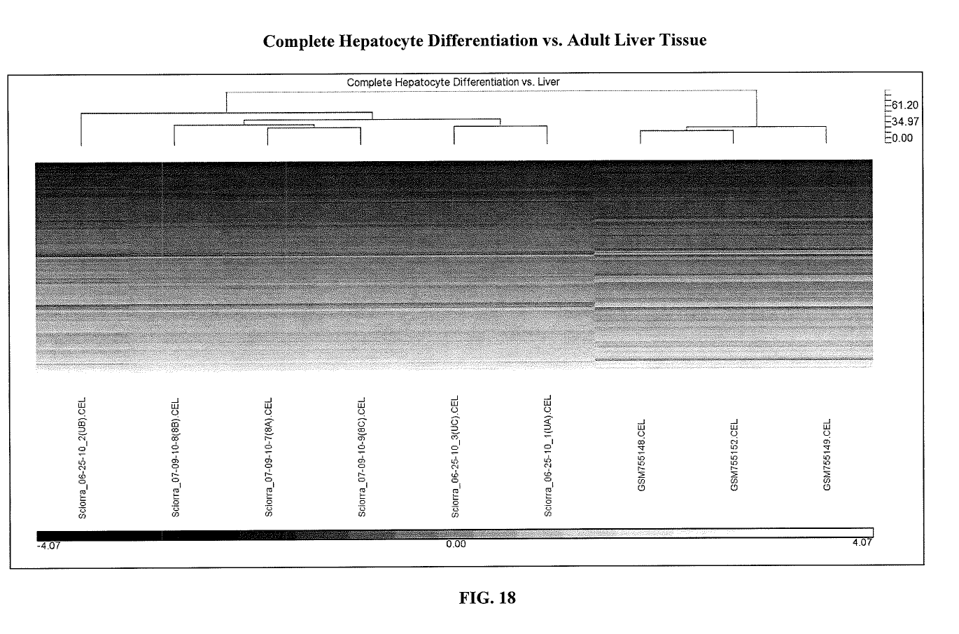

Lastly, FIG. 17 is a collection of data showing a heat map and clustering diagram of the entire genome, comparing the gene expression of fibroblast-derived multipotent stem cells, differentiated fibroblast-derived adipose tissue from the 3 patients, and adult lean adipose tissue samples, and adult obese adipose tissue samples. Similarly, FIG. 18 is a collection of data showing a heat map and clustering diagram of the entire genome, comparing the gene expression of fibroblast-derived multipotent stem cells, differentiated fibroblast-derived hepatic tissue from the 3 patients, and adult hepatic tissue samples.

Collectively, these data show that from a gene expression profile perspective, the differentiated lineage-specific tissue generated from fibroblasts closely resemble those of actual tissue from that lineage. This is true for adipose and hepatic tissues. Thus, the data presented in this Example suggests that multipotent stem cells produced by the disclosed methods are capable of differentiation into cells of any of the 3 germ layers and that in terms of gene expression, the fibroblast-derived differentiated multipotent stem cells approximate normal differentiated tissue.

### EXAMPLE 6

DNA from fibroblast-derived multipotent stem cells from the 3 patients at passage 3 was analyzed by karyotyping and by comparative genomic hybridization (CGH). Results from both analyses show that the fibroblast-derived multipotent stem cells do not exhibit an increased rate of mutations (data not shown). These results indicate that the methods described herein for propagation of multipotent stem cells does not increase the rate of mutations, suggesting the safety of the multipotent stem cells.

### EXAMPLE 7

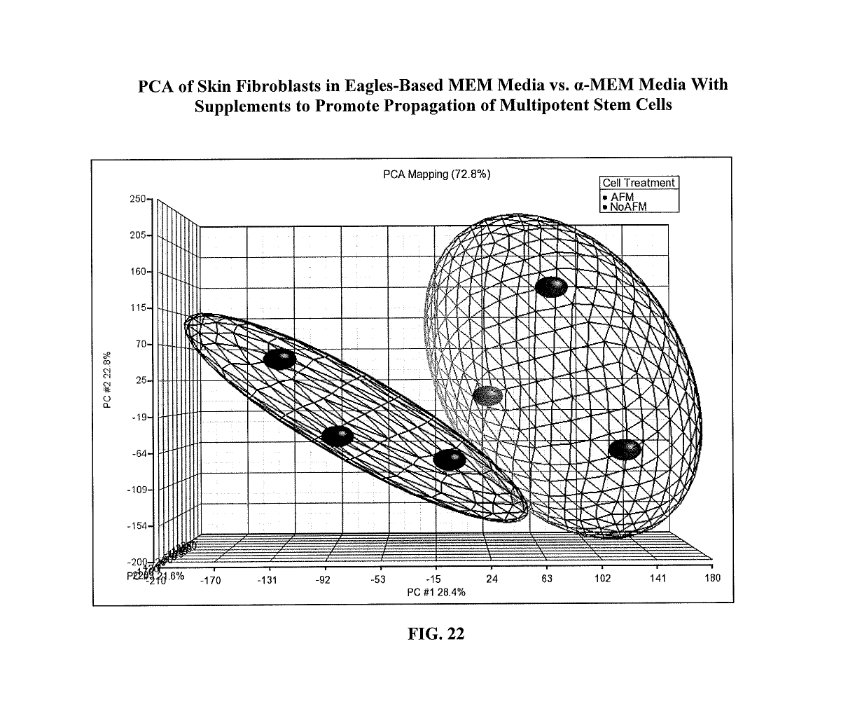

Skin fibroblasts from 3 patients of varying ages were subject to PCA analysis at two different time points: (1) while culturing in standard conventional media (Eagles-based MEM media); and (2) while culturing for 3 passages in media that promotes propagation of multipotent stem cells (α-MEM media plus supplements). The PCA, shown in FIG. 22, detects gene changes that occur during the transfer in media. These studies show that the 3 patient samples appear to cluster together as cells under the 3 passages in media that promotes propagation of multipotent stem cells.

### EXAMPLE 8

A 3-dimensional tissue model was generated and used to identify differentially expressed genes during hepatic differentiation, using lamin bead plates as a 3-dimensional scaffold or matrix for cellular growth and differentiation.

Laminin-Coated Bead Plates

The 3-dimensional tissue model was based on the use of plates with laminin-coated beads, or laminin-coated bead plates. These laminin-coated bead plates were generated by first obtaining a stock substrate of laminin at a concentration of 1 mg/ml dissolved in cold Phosphate Buffered Saline (PBS). Roughly 1 ml of this stock substrate of laminin was added to each well of a 6-well Falcon Tissue Culture plate. Spherical glass beads (Sartorius, Inc.) were then obtained and allowed to sit under ultraviolet (UV) excitation for sterilization. In this experiment, a mixture of beads (20-25 micron diameter (66.7%) and 17-20 micron diameter (33.3%)) was employed. After the roughly 1 ml of laminin was added to the wells, beads were added to the laminin. The plate was then placed in an incubator at 37° C. for 12 hours in order to induce polymerization of the laminin. After this incubation period, the plate, any excess PBS was drawn off using a sterile pipette, and the plate was allowed to completely air dry.

Fibroblast-Derived Multipotent Stem Cells.

Fibroblast-derived multipotent stem cells from 3 patients were generated as described in Example 1 (i.e., in media comprising amniotic medium comprising amniotic growth fluid media (AFM) (as described in DeCoppi et al., comprising α-MEM (Invitrogen), 15% ES-FBS (Invitrogen), 1% L-Glutamine, and 1% Pen/Strep, supplemented with 18% CHANG MEDIUM® B (Irvine Scientific) and 2% CHANG MEDIUM® C (Irvine Scientific)), so as to propagate multipotent stem cells, as described in International Patent Publication No. WO 2009/151844). The AFM comprises α-MEM media plus supplements.

3-Dimensional Tissue Model

These cells were then plated onto the laminin-coated bead plates at a concentration of 5,000 cells/cm² and then placed back into the 37° C. incubator at an atmosphere of 5% CO₂ for three days. The media was then removed from wells containing the fibroblast-derived multipotent stem cells in laminin-coated bead plates, and was replaced with culture media conditioned to promote hepatic differentiation. A coverslip (about 8 cm²) was then placed atop the cells in the wells of the laminin-coated bead plate, which created a 3-dimensional space for the cells to then grown and differentiate. Culture media from control (undifferentiated) wells was replaced again with media comprising amniotic medium comprising amniotic growth fluid media (AFM). The AFM comprises α-MEM media plus supplements.

For cells undergoing hepatic differentiation, fresh hepatic differentiation culture media was replaced every three days by gently removing old culture media and adding fresh new culture media. By contrast, undifferentiated control multipotent stem cells were given fresh media comprising amniotic medium comprising amniotic growth fluid media (AFM). The AFM comprises α-MEM media plus supplements.

Cells grown in the hepatic differentiation media on laminin-coated bead plates and undifferentiated control multipotent stem cells were harvested at days 1, 3, 6, 10, 12, 17, 25, 33, 38, and 45 for microarray analysis. The microarray analysis was conducted to assess the gene expression changes over this period of time in the 3-dimensional model of differentiating hepatic cells as compared to control undifferentiated cells. Microarray analysis was conducted as described in International Patent Publication No. WO 2009/151844

The microarray results are shown in tabular format for hepatic differentiation after day 1 (Table 1), day 3 (Table 2), day 6 (Table 3), day 10 (Table 4), day 12 (Table 5), day 17 (Table 6), day 25 (Table 7), day 33 (Table 8), day 38 (Table 9), and day 45 (Table 10) for each of 3 individuals. Each individual is designated by their identification numbers, 731, 970, and 1650. These data show that individual 731, 970, and 1650 each had 93, 88, and 82 genes, respectively, which were up- or down-regulated 15-fold for at least two time points. Taken together, these studies show that there are a number of common genes that are differentially regulated across individuals during hepatic differentiation. These common genes may provide insight into genes that are important in hepatic differentiation and hepatic biology and function.

TABLE 1
Gene expression profiles for samples from three individuals (identified by
numbers 731, 970, and 1650) of hepatic differentiated (day 1) vs. undifferentiated
cell types. All genes displaying at least 2-fold up- or down-regulation common
in all three individuals are shown.

| | | | | Fold- | |
|---|---|---|---|---|---|
| Probe | | mRNA | Fold-Change | Change | Fold-Change |
| Set ID | Gene Symbol | Accession | (731) | (970) | (1650) |
| 8083594 | PTX3 | NM_002852 | 25.7028 | 15.0749 | 9.24006 |
| 7995787 | MT1M | NM_176870 | 21.8199 | 18.302 | 17.1208 |
| 8025402 | ANGPTL4 | NM_139314 | 15.8633 | 7.35803 | 9.14268 |
| 7934979 | ANKRD1 | NM_014391 | 12.8752 | 5.14143 | 14.4051 |
| 8125919 | FKBP5 | NM_001145775 | 10.4985 | 5.17476 | 12.0802 |
| 8142270 | NRCAM | NM_001193582 | 10.1569 | 4.73963 | 13.8673 |
| 7964834 | CPM | NM_001874 | 8.22423 | 3.4351 | 3.6459 |
| 8047926 | MAP2 | NM_002374 | 7.74427 | 4.76553 | 4.94333 |
| 8095744 | AREG | NM_001657 | 6.7058 | 2.42966 | 4.58167 |
| 7928308 | DDIT4 | NM_019058 | 6.61344 | 8.725 | 2.4703 |
| 7962183 | AK4 | NM_001005353 | 5.17857 | 2.01707 | 2.50487 |
| 8140556 | HGF | NM_000601 | 3.32531 | 2.23157 | 3.06731 |
| 8095736 | AREG | NM_001657 | 3.18003 | 2.01508 | 2.56961 |
| 8120838 | TTK | NM_003318 | −2.09017 | −2.57179 | −2.07086 |
| 8124537 | HIST1H3J | NM_003535 | −2.10671 | −2.31392 | −2.1088 |
| 8054580 | BUB1 | NM_004336 | −2.14724 | −2.21585 | −2.41877 |
| 7929258 | KIF11 | NM_004523 | −2.16012 | −2.51438 | −2.78451 |
| 7909708 | CENPF | NM_016343 | −2.20973 | −2.27734 | −2.26709 |
| 8014974 | TOP2A | NM_001067 | −2.26251 | −2.36371 | −2.52757 |
| 8132318 | ANLN | NM_018685 | −2.28202 | −2.13796 | −2.35139 |
| 8108301 | KIF20A | NM_005733 | −2.3319 | −2.36191 | −2.37569 |
| 7947199 | LGR4 | NM_018490 | −2.36058 | −2.16172 | −5.77244 |
| 7982889 | NUSAP1 | NM_016359 | −2.3873 | −2.77451 | −2.37489 |
| 7969243 | CKAP2 | NM_018204 | −2.43552 | −2.65892 | −2.46081 |
| 7917255 | SSX2IP | NM_014021 | −2.4554 | −3.49591 | −3.04079 |
| 8095585 | SLC4A4 | NM_001098484 | −2.49169 | −2.26387 | −3.18114 |
| 8151101 | MYBL1 | NM_001080416 | −3.06675 | −3.94656 | −3.77018 |
| 7923086 | ASPM | NM_018136 | −3.23469 | −3.68736 | −3.54465 |
| 7971104 | TRPC4 | NM_016179 | −3.55001 | −4.13931 | −5.15296 |
| 7976567 | BDKRB1 | NM_000710 | −4.20844 | −3.6416 | −5.43657 |
| 8015349 | KRT19 | NM_002276 | −4.51085 | −3.52882 | −4.24316 |

TABLE 2
Gene expression profiles for samples from three individuals (identified by
numbers 731, 970, and 1650) of hepatic differentiated (day 3) vs. undifferentiated cell
types. All genes displaying at least 3-fold up- or down-regulation common in all three
individuals are shown.

| | | | | Fold- | |
|---|---|---|---|---|---|
| Probe | | mRNA | Fold-Change | Change | Fold-Change |
| Set ID | Gene Symbol | Accession | (731) | (970) | (1650) |
| 8083594 | PTX3 | NM_002852 | 30.3395 | 20.8435 | 7.60548 |
| 7995787 | MT1M | NM_176870 | 5.2118 | 4.5535 | 5.46458 |
| 8025402 | ANGPTL4 | NM_139314 | 5.90102 | 3.49589 | 3.60523 |
| 8125919 | FKBP5 | NM_001145775 | 9.11861 | 4.9441 | 15.3706 |
| 7964834 | CPM | NM_001874 | 19.4435 | 5.57631 | 9.17289 |
| 7928308 | DDIT4 | NM_019058 | 12.4065 | 16.279 | 4.29295 |
| 8092970 | APOD | NM_001647 | 46.9117 | 8.41138 | 18.9768 |
| 8151871 | CCNE2 | NM_057749 | −3.78611 | −9.62346 | −3.90021 |
| 7960744 | C1R | NM_001733 | 8.13993 | 4.75303 | 3.91245 |
| 8135915 | C7orf68 | NM_013332 | 6.81017 | 7.33263 | 4.94534 |
| 8133106 | SNORA22 | NR_002961 | 3.68625 | 4.67124 | 5.25726 |
| 7935776 | SCD | NM_005063 | 3.04662 | 5.23739 | 3.25807 |
| 8096875 | ENPEP | NM_001977 | 4.09927 | 5.57169 | 5.52659 |
| 7914342 | FABP3 | NM_004102 | 5.36206 | 4.89292 | 3.5376 |
| 8130578 | SNORA20 | NR_002960 | 3.76981 | 3.17257 | 3.05955 |
| 8014063 | EVI2B | NM_006495 | 8.39167 | 4.22062 | 7.83691 |
| 8147516 | MATN2 | NM_002380 | −5.25162 | −3.69195 | −5.08239 |
| 8120654 | KCNQ5 | NM_001160133 | −4.30264 | −6.65369 | −4.80569 |
| 8135909 | LEP | NM_000230 | 8.92851 | 10.5553 | 7.91317 |
| 8174598 | IL13RA2 | NM_000640 | −3.71277 | −3.3302 | −3.69767 |
| 8060813 | MCM8 | NM_032485 | −4.29108 | −5.69793 | −3.47919 |
| 7916898 | DEPDC1 | NM_001114120 | −4.80442 | −6.57943 | −3.28586 |
| 7951284 | MMP3 | NM_002422 | −7.78401 | −5.32783 | −6.76082 |
| 8142981 | PODXL | NM_001018111 | −14.1465 | −12.3641 | −6.82389 |
| 8135601 | MET | NM_001127500 | −5.16536 | −3.98395 | −3.71986 |
| 7952785 | OPCML | NM_001012393 | −6.791 | −17.9216 | −4.37607 |
| 8145570 | ESCO2 | NM_001017420 | −5.98133 | −10.5938 | −3.90002 |
| 7927710 | CDK1 | NM_001786 | −4.1992 | −7.69563 | −3.11625 |
| 8092177 | NCEH1 | NM_001146276 | −5.01826 | −6.07116 | −3.58301 |
| 8046380 | ITGA6 | NM_000210 | −16.2581 | −9.85997 | −11.7434 |
| 7984540 | KIF23 | NM_138555 | −6.04761 | −5.69183 | −3.35572 |
| 8085138 | OXTR | NM_000916 | −8.44601 | −6.57678 | −5.00445 |
| 7947199 | LGR4 | NM_018490 | −3.22619 | −3.18139 | −6.25298 |
| 7917255 | SSX2IP | NM_014021 | −6.28932 | −8.04475 | −5.49439 |
| 8095585 | SLC4A4 | NM_001098484 | −7.06149 | −6.99498 | −7.35258 |
| 8151101 | MYBL1 | NM_001080416 | −6.79619 | −10.2036 | −7.42154 |
| 7923086 | ASPM | NM_018136 | −6.62611 | −8.59572 | −3.58084 |
| 7971104 | TRPC4 | NM_016179 | −5.3725 | −4.47693 | −5.68616 |
| 7987315 | ACTC1 | NM_005159 | −13.1087 | −10.4742 | −23.0797 |
| 7976567 | BDKRB1 | NM_000710 | −11.3348 | −11.1869 | −8.66277 |
| 8015349 | KRT19 | NM_002276 | −8.5028 | −6.84666 | −6.31428 |

TABLE 3
Gene expression profiles for samples from three individuals (identified by
numbers 731, 970, and 1650) of hepatic differentiated (day 6) vs. undifferentiated cell
types. All genes displaying at least 10-fold up- or down-regulation common in
all three individuals are shown.

| | | | | Fold- | |
|---|---|---|---|---|---|
| Probe | | mRNA | Fold-Change | Change | Fold-Change |
| Set ID | Gene Symbol | Accession | (731) | (970) | (1650) |
| 8092970 | APOD | NM_001647 | 63.6401 | 32.1907 | 33.09 |
| 7914342 | FABP3 | NM_004102 | 20.5176 | 31.3482 | 10.1749 |
| 7909568 | DTL | NM_016448 | −11.8651 | −14.0892 | −11.2525 |
| 8060813 | MCM8 | NM_032485 | −10.1964 | −10.5175 | −12.7576 |
| 7916898 | DEPDC1 | NM_001114120 | −17.2463 | −23.3744 | −20.2521 |
| 8040223 | RRM2 | NM_001165931 | −10.9578 | −11.804 | −11.1433 |
| 8142981 | PODXL | NM_001018111 | −26.014 | −22.5631 | −19.6088 |
| 8021187 | SKA1 | NM_001039535 | −12.5167 | −14.6301 | −12.4346 |
| 7970513 | SKA3 | NM_145061 | −11.5356 | −11.3527 | −13.3158 |
| 8001133 | SHCBP1 | NM_024745 | −15.6335 | −17.6245 | −15.4674 |
| 8117594 | HIST1H2BM | NM_003521 | −19.0157 | −14.5016 | −17.2419 |
| 8056572 | SPC25 | NM_020675 | −12.9835 | −15.6141 | −10.6873 |
| 8145570 | ESCO2 | NM_001017420 | −27.2039 | −21.5102 | −25.8602 |
| 7927710 | CDK1 | NM_001786 | −13.8693 | −22.464 | −20.7863 |
| 8094278 | NCAPG | NM_022346 | −13.0486 | −16.7803 | −15.9578 |
| 8046380 | ITGA6 | NM_000210 | −28.7284 | −17.5904 | −22.4716 |
| 7929334 | CEP55 | NM_018131 | −20.1806 | −20.4144 | −16.0893 |
| 8054702 | CKAP2L | NM_152515 | −10.5867 | −18.0606 | −12.5419 |
| 8085754 | SGOL1 | NM_001012410 | −14.0387 | −16.4692 | −17.861 |
| 8124388 | HIST1H3B | NM_003537 | −16.2453 | −15.1203 | −10.6307 |
| 7982757 | CASC5 | NM_170589 | −16.5128 | −23.3947 | −19.4925 |
| 8061579 | TPX2 | NM_012112 | −14.7729 | −15.1859 | −15.5952 |
| 7937020 | MKI67 | NM_002417 | −10.862 | −13.9273 | −11.1542 |
| 7974404 | CDKN3 | NM_005192 | −17.9285 | −23.418 | −21.2436 |
| 7906930 | NUF2 | NM_145697 | −24.1246 | −20.6648 | −20.6703 |
| 8120838 | TTK | NM_003318 | −17.0286 | −28.0153 | −15.2684 |
| 7983969 | CCNB2 | NM_004701 | −14.0894 | −15.2591 | −13.5907 |
| 8054580 | BUB1 | NM_004336 | −13.4537 | −17.7715 | −14.1001 |
| 7929258 | KIF11 | NM_004523 | −14.511 | −15.265 | −12.9951 |
| 7909708 | CENPF | NM_016343 | −10.5236 | −12.817 | −11.5195 |
| 8102643 | CCNA2 | NM_001237 | −12.2259 | −14.9277 | −10.6569 |
| 8014974 | TOP2A | NM_001067 | −12.9532 | −17.4024 | −12.5757 |
| 8109712 | HMMR | NM_001142556 | −11.8044 | −10.9385 | −11.4646 |
| 7984540 | KIF23 | NM_138555 | −14.7154 | −17.1207 | −15.1236 |
| 8132318 | ANLN | NM_018685 | −21.2876 | −20.1581 | −20.0911 |
| 7900699 | CDC20 | NM_001255 | −22.4067 | −14.9763 | −14.7787 |
| 8108301 | KIF20A | NM_005733 | −20.83 | −25.5036 | −22.3207 |
| 8149955 | PBK | NM_018492 | −16.1135 | −19.6492 | −13.5689 |
| 7982889 | NUSAP1 | NM_016359 | −10.9847 | −15.6213 | −11.5089 |
| 7994109 | PLK1 | NM_005030 | −14.5652 | −12.1821 | −13.2616 |
| 8151101 | MYBL1 | NM_001080416 | −10.36 | −13.739 | −15.2515 |
| 7979307 | DLGAP5 | NM_014750 | −27.0698 | −28.2352 | −15.785 |
| 7923086 | ASPM | NM_018136 | −29.3922 | −34.6268 | −23.3511 |
| 7987315 | ACTC1 | NM_005159 | −11.9448 | −12.6572 | −28.2205 |
| 7976567 | BDKRB1 | NM_000710 | −13.9694 | −14.1704 | −10.7645 |

TABLE 4
Gene expression profiles for samples from three individuals (identified by
numbers 731, 970, and 1650) of hepatic differentiated (day 10) vs. undifferentiated
cell types. All genes displaying at least 10-fold up- or down-regulation common
in all three individuals are shown.

| | | | | Fold- | |
|---|---|---|---|---|---|
| Probe | | mRNA | Fold-Change | Change | Fold-Change |
| Set ID | Gene Symbol | Accession | (731) | (970) | (1650) |
| 8092970 | APOD | NM_001647 | 68.9133 | 41.7707 | 35.2446 |
| 8141016 | TFPI2 | NM006528 | −15.6828 | −11.0953 | −19.6027 |
| 7914342 | FABP3 | NM_004102 | 32.9292 | 51.1672 | 22.0864 |
| 8130578 | SNORA20 | NR_002960 | 13.1713 | 12.3668 | 18.7426 |
| 8060813 | MCM8 | NM_032485 | −13.0632 | −10.0243 | −13.1601 |
| 7916898 | DEPDC1 | NM_001114120 | −23.4374 | −19.9358 | −15.688 |
| 8097356 | PLK4 | NM_014264 | −15.5163 | −12.3037 | −15.664 |
| 8142981 | PODXL | NM_001018111 | −32.2909 | −25.8501 | −16.2459 |
| 8021187 | SKA1 | NM_001039535 | −13.6957 | −11.8173 | −10.3903 |
| 7923189 | KIF14 | NM_014875 | −13.8822 | −10.5022 | −11.7232 |
| 8001133 | SHCBP1 | NM_024745 | −18.156 | −12.8266 | −13.975 |
| 8117594 | HIST1H2BM | NM_003521 | −25.1964 | −10.6829 | −15.0303 |
| 8056572 | SPC25 | NM_020675 | −11.5759 | −11.9497 | −11.9084 |
| 8145570 | ESCO2 | NM_001017420 | −46.7107 | −17.1167 | −28.0824 |
| 7927710 | CDK1 | NM_001786 | −23.6868 | −15.9595 | −19.5659 |
| 8094278 | NCAPG | NM_022346 | −16.4622 | −14.2592 | −13.266 |
| 8046380 | ITGA6 | NM_000210 | −34.8909 | −22.8495 | −22.6072 |
| 7929334 | CEP55 | NM_018131 | −17.743 | −11.7215 | −18.6339 |
| 8054702 | CKAP2L | NM_152515 | −12.228 | −10.3821 | −10.4132 |
| 8085754 | SGOL1 | NM_001012410 | −16.8883 | −13.0281 | −14.6698 |
| 7982757 | CASC5 | NM_170589 | −27.7392 | −14.0897 | −18.8298 |
| 8061579 | TPX2 | NM_012112 | −19.7956 | −10.0017 | −13.5713 |
| 7974404 | CDKN3 | NM_005192 | −28.3035 | −20.3289 | −21.6472 |
| 7906930 | NUF2 | NM_145697 | −16.7132 | −11.1472 | −18.7732 |
| 8120838 | TTK | NM_003318 | −21.2318 | −15.547 | −18.2735 |
| 7983969 | CCNB2 | NM_004701 | −20.6167 | −12.0802 | −12.6952 |
| 8054580 | BUB1 | NM_004336 | −18.9961 | −11.8212 | −19.9417 |
| 7929258 | KIF11 | NM_004523 | −19.6767 | −11.8609 | −14.6221 |
| 7984540 | KIF23 | NM_138555 | −18.1939 | −11.6047 | −13.1659 |
| 8132318 | ANLN | NM_018685 | −27.7326 | −11.6962 | −19.1003 |
| 7900699 | CDC20 | NM_001255 | −26.875 | −13.2593 | −13.6862 |
| 8108301 | KIF20A | NM_005733 | −27.1896 | −17.5099 | −21.2622 |
| 8149955 | PBK | NM_018492 | −21.0555 | −11.7959 | −11.9119 |
| 7982889 | NUSAP1 | NM_016359 | −16.63 | −12.7869 | −11.7961 |
| 7994109 | PLK1 | NM_005030 | −15.7272 | −11.0327 | −11.6765 |
| 8095585 | SLC4A4 | NM_001098484 | −17.6935 | −10.2597 | −10.8457 |
| 7979307 | DLGAP5 | NM_014750 | −29.9358 | −11.7283 | −16.6535 |
| 7923086 | ASPM | NM_018136 | −37.1396 | −17.3282 | −21.189 |
| 7987315 | ACTC1 | NM_005159 | −12.8655 | −11.1982 | −29.4309 |
| 7976567 | BDKRB1 | NM_000710 | −19.098 | −12.0674 | −12.1562 |

TABLE 5
Gene expression profiles for samples from three individuals (identified by
numbers 731, 970, and 1650) of hepatic differentiated (day 12) vs. undifferentiated
cell types. All genes displaying at least 10-fold up- or down-regulation common
in all three individuals are shown.

| | | | | Fold- | |
|---|---|---|---|---|---|
| Probe | | mRNA | Fold-Change | Change | Fold-Change |
| Set ID | Gene Symbol | Accession | (731) | (970) | (1650) |
| 8092970 | APOD | NM_001647 | 62.3856 | 41.3006 | 37.7508 |
| 8133106 | SNORA22 | NR _002961 | 15.8875 | 11.3384 | 21.3107 |
| 8162394 | ASPN | NM_017680 | 20.7057 | 10.0278 | 20.2447 |
| 8162388 | OMD | NM_005014 | 13.8717 | 17.6793 | 10.3657 |
| 7914342 | FABP3 | NM_004102 | 16.965 | 48.1936 | 25.0974 |
| 8130578 | SNORA20 | NR_002960 | 23.3477 | 18.1911 | 29.5969 |
| 7977507 | RPPH1 | NR_002312 | 13.2224 | 13.5778 | 13.8652 |
| 8009380 | SNORA38B | NR_003706 | 10.8544 | 10.1163 | 14.0914 |
| 8135909 | LEP | NM_000230 | 18.4188 | 16.976 | 13.721 |
| 8023392 | SNORA37 | NR _002970 | 13.8946 | 12.0238 | 14.4715 |
| 7938329 | SNORA23 | NR _002962 | 15.9444 | 15.3704 | 16.8229 |
| 7916898 | DEPDC1 | NM_001114120 | −11.5808 | −17.1003 | −14.3503 |
| 8097356 | PLK4 | NM_014264 | −11.6892 | −14.4548 | −11.6182 |
| 8142981 | PODXL | NM_001018111 | −25.3486 | −28.3843 | −20.1403 |
| 8001133 | SHCBP1 | NM_024745 | −14.0914 | −16.9758 | −11.2108 |
| 8117594 | HIST1H2BM | NM_003521 | −18.6634 | −13.8923 | −12.9928 |
| 8145570 | ESCO2 | NM_001017420 | −22.0196 | −16.3605 | −20.4903 |
| 7927710 | CDK1 | NM_001786 | −15.1084 | −17.8366 | −12.4258 |
| 8094278 | NCAPG | NM_022346 | −14.3838 | −14.0245 | −10.1197 |
| 8046380 | ITGA6 | NM_000210 | −32.6746 | −30.2244 | −21.0493 |
| 7929334 | CEP55 | NM_018131 | −15.454 | −10.885 | −10.1603 |
| 8085754 | SGOL1 | NM_001012410 | −12.3814 | −10.1293 | −12.3605 |
| 7982757 | CASC5 | NM_170589 | −17.3203 | −14.7567 | −13.371 |
| 7974404 | CDKN3 | NM_005192 | −18.6409 | −15.725 | −11.9337 |
| 8054580 | BUB1 | NM_004336 | −15.4831 | −11.5552 | −10.9449 |
| 7929258 | KIF11 | NM_004523 | −16.9821 | −15.356 | −10.986 |
| 8132318 | ANLN | NM_018685 | −16.1389 | −10.5164 | −10.2032 |
| 8108301 | KIF20A | NM_005733 | −16.0288 | −14.2485 | −13.5796 |
| 8095585 | SLC4A4 | NM_001098484 | −13.7244 | −12.8051 | −10.045 |
| 7979307 | DLGAP5 | NM_014750 | −22.4725 | −14.2664 | −10.3782 |
| 7923086 | ASPM | NM_018136 | −25.3499 | −17.7753 | −12.9735 |
| 7987315 | ACTC1 | NM_005159 | −15.1603 | −12.5897 | −31.8732 |
| 7976567 | BDKRB1 | NM_000710 | −11.9456 | −16.9748 | −13.8472 |

TABLE 6
Gene expression profiles for samples from three individuals (identified by
numbers 731, 970, and 1650) of hepatic differentiated (day 17) vs. undifferentiated
cell types. All genes displaying at least 12-fold up- or down-regulation common
in all three individuals are shown.

| | | | | Fold- | |
|---|---|---|---|---|---|
| Probe | | mRNA | Fold-Change | Change | Fold-Change |
| Set ID | Gene Symbol | Accession | (731) | (970) | (1650) |
| 8092970 | APOD | NM_001647 | 57.2413 | 37.5961 | 29.7746 |
| 8133106 | SNORA22 | NR_002961 | 12.2679 | 12.6297 | 19.3902 |
| 7998666 | SNORA64 | NR_002326 | 13.27 | 12.2299 | 19.3847 |
| 8162394 | ASPN | NM_017680 | 23.6156 | 28.3394 | 24.3414 |
| 7920873 | SNORA42 | NR_002974 | 26.3843 | 14.7094 | 26.3892 |
| 7914342 | FABP3 | NM_004102 | 13.1984 | 26.4812 | 13.2272 |
| 8130578 | SNORA20 | NR_002960 | 38.121 | 35.9495 | 54.1602 |
| 7977507 | RPPH1 | NR_002312 | 18.6574 | 16.0082 | 14.7236 |
| 8009380 | SNORA38B | NR_003706 | 23.1752 | 20.9736 | 29.8509 |
| 8135909 | LEP | NM_000230 | 20.4969 | 23.4281 | 14.3679 |
| 8049299 | SCARNA6 | NR_003006 | 12.6607 | 14.4461 | 15.3659 |
| 8023392 | SNORA37 | NR_002970 | 20.4348 | 18.402 | 25.9976 |
| 7938329 | SNORA23 | NR_002962 | 19.9244 | 21.3623 | 27.442 |
| 7916898 | DEPDC1 | NM_001114120 | −20.2062 | −14.7758 | −18.5392 |
| 8142981 | PODXL | NM_001018111 | −42.5689 | −29.7177 | −17.713 |
| 8001133 | SHCBP1 | NM_024745 | −18.6412 | −18.8208 | −13.4745 |
| 8117594 | HIST1H2BM | NM_003521 | −28.0813 | −13.6371 | −17.919 |
| 8145570 | ESCO2 | NM_001017420 | −34.0606 | −26.3714 | −27.8274 |
| 7927710 | CDK1 | NM_001786 | −14.5077 | −16.2072 | −15.8429 |
| 8046380 | ITGA6 | NM_000210 | −41.0972 | −32.0706 | −24.6844 |
| 7929334 | CEP55 | NM_018131 | −16.5164 | −16.1579 | −13.0563 |
| 8085754 | SGOL1 | NM_001012410 | −13.876 | −15.8975 | −15.7542 |
| 7982757 | CASC5 | NM_170589 | −18.9653 | −15.34 | −15.1125 |
| 8061579 | TPX2 | NM_012112 | −14.8453 | −13.1334 | −12.2978 |
| 7974404 | CDKN3 | NM_005192 | −23.8829 | −19.48 | −20.0499 |
| 7929258 | KIF11 | NM_004523 | −16.8152 | −12.3758 | −13.6087 |
| 8132318 | ANLN | NM_018685 | −20.1714 | −12.7609 | −13.1742 |
| 8108301 | KIF20A | NM_005733 | −18.7512 | −16.2361 | −17.0865 |
| 7923086 | ASPM | NM_018136 | −24.3799 | −19.9013 | −19.6574 |
| 7987315 | ACTC1 | NM_005159 | −14.9801 | −15.7117 | −29.031 |

TABLE 7
Gene expression profiles for samples from three individuals (identified by
numbers 731, 970, and 1650) of hepatic differentiated (day 25) vs. undifferentiated
cell types. All genes displaying at least 15-fold up- or down-regulation common
in all three individuals are shown.

| | | | | Fold- | |
|---|---|---|---|---|---|
| Probe | | mRNA | Fold-Change | Change | Fold-Change |
| Set ID | Gene Symbol | Accession | (731) | (970) | (1650) |
| 8092970 | APOD | NM_001647 | 64.2504 | 74.0558 | 32.3365 |
| 8133106 | SNORA22 | NR_002961 | 16.9289 | 18.5087 | 26.1679 |
| 7920873 | SNORA42 | NR_002974 | 35.365 | 15.649 | 30.5986 |
| 8130578 | SNORA20 | NR_002960 | 43.5177 | 32.8465 | 48.4411 |
| 7977507 | RPPH1 | NR_002312 | 18.3033 | 17.1418 | 16.7277 |
| 7916898 | DEPDC1 | NM_001114120 | −23.0266 | −37.4312 | −17.6386 |
| 7970513 | SKA3 | NM_145061 | −23.8512 | −26.1033 | −22.0416 |
| 7929078 | KIF20B | NM_016195 | −19.5287 | −19.0186 | −15.2284 |
| 7989647 | KIAA0101 | NM_014736 | −19.2882 | −23.5699 | −15.8652 |
| 8001133 | SHCBP1 | NM_024745 | −22.3111 | −26.2348 | −16.266 |
| 8117594 | HIST1H2BM | NM_003521 | −50.1791 | −40.0168 | −16.4013 |
| 8145570 | ESCO2 | NM_001017420 | −37.356 | −29.9863 | −19.7688 |
| 7927710 | CDK1 | NM_001786 | −20.1699 | −42.2377 | −15.7257 |
| 8046380 | ITGA6 | NM_000210 | −37.6923 | −33.3884 | −33.8703 |
| 7929334 | CEP55 | NM_018131 | −32.133 | −27.8375 | −18.3601 |
| 7982757 | CASC5 | NM_170589 | −40.9138 | −52.5331 | −29.0436 |
| 8061579 | TPX2 | NM_012112 | −32.7446 | −31.5596 | −23.0918 |
| 7974404 | CDKN3 | NM_005192 | −29.4267 | −30.9295 | −28.931 |
| 8120838 | TTK | NM_003318 | −28.5384 | −41.2909 | −16.6636 |
| 7983969 | CCNB2 | NM_004701 | −24.1983 | −30.2779 | −17.7251 |
| 8054580 | BUB1 | NM_004336 | −26.5822 | −26.3303 | −16.2427 |
| 7929258 | KIF11 | NM_004523 | −24.523 | −28.0516 | −19.1474 |
| 7984540 | KIF23 | NM_138555 | −22.105 | −17.2037 | −15.7121 |
| 8132318 | ANLN | NM_018685 | −67.7282 | −54.6324 | −24.4251 |
| 8108301 | KIF20A | NM_005733 | −53.5901 | −66.83 | −43.9804 |
| 8149955 | PBK | NM_018492 | −27.9869 | −41.2947 | −18.237 |
| 7979307 | DLGAP5 | NM_014750 | −57.8301 | −41.697 | −30.7483 |
| 7923086 | ASPM | NM_018136 | −48.194 | −66.0815 | −35.0628 |

TABLE 8
Gene expression profiles for samples from three individuals (identified by
numbers 731, 970, and 1650) of hepatic differentiated (day 33) vs. undifferentiated
cell types. All genes displaying at least 15-fold up- or down-regulation common
in all three individuals are shown.

| | | | | Fold- | |
|---|---|---|---|---|---|
| Probe | | mRNA | Fold-Change | Change | Fold-Change |
| Set ID | Gene Symbol | Accession | (731) | (970) | (1650) |
| 8095744 | AREG | NM_001657 | 23.8166 | 15.3452 | 18.2455 |
| 8092970 | APOD | NM_001647 | 49.3954 | 50.9372 | 34.2286 |
| 8133106 | SNORA22 | NR_002961 | 20.9427 | 15.3904 | 23.7516 |
| 8162394 | ASPN | NM_017680 | 37.4194 | 29.2956 | 21.9551 |
| 7914342 | FABP3 | NM_004102 | 23.8968 | 43.7663 | 48.7613 |
| 7909568 | DTL | NM_016448 | −22.3656 | −16.1311 | −18.4669 |
| 8130578 | SNORA20 | NR_002960 | 53.0306 | 29.4053 | 41.2016 |
| 8135909 | LEP | NM_000230 | 27.131 | 45.0239 | 20.179 |
| 8142981 | PODXL | NM_001018111 | −39.1376 | −35.996 | −20.9764 |
| 8001133 | SHCBP1 | NM_024745 | −19.9596 | −23.0012 | −30.3738 |
| 8117594 | HIST1H2BM | NM_003521 | −48.9424 | −25.7049 | −23.4044 |
| 8145570 | ESCO2 | NM_001017420 | −35.5439 | −29.557 | −26.5657 |
| 7927710 | CDK1 | NM_001786 | −21.7789 | −26.8093 | −29.9973 |
| 8094278 | NCAPG | NM_022346 | −28.1642 | −20.0178 | −22.835 |
| 8046380 | ITGA6 | NM_000210 | −57.4916 | −27.3304 | −24.2961 |
| 7929334 | CEP55 | NM_018131 | −28.7033 | −20.8262 | −23.4344 |
| 8085754 | SGOL1 | NM_001012410 | −20.1815 | −17.5419 | −20.9295 |
| 8124388 | HIST1H3B | NM_003537 | −20.3601 | −17.2877 | −19.8258 |
| 7982757 | CASC5 | NM_170589 | −24.0403 | −29.1231 | −26.848 |
| 8061579 | TPX2 | NM_012112 | −31.4346 | −16.3829 | −17.7183 |
| 7937020 | MKI67 | NM_002417 | −18.4026 | −16.2738 | −18.5837 |
| 7974404 | CDKN3 | NM_005192 | −28.0183 | −27.427 | −36.0525 |
| 7906930 | NUF2 | NM_145697 | −25.7027 | −19.6636 | −21.7887 |
| 8120838 | TTK | NM_003318 | −17.7973 | −23.8381 | −26.1016 |
| 7983969 | CCNB2 | NM_004701 | −23.5458 | −18.604 | −19.7292 |
| 8054580 | BUB1 | NM_004336 | −32.8398 | −16.1866 | −17.9084 |
| 7929258 | KIF11 | NM_004523 | −29.1859 | −22.7685 | −25.4079 |
| 8014974 | TOP2A | NM_001067 | −24.4816 | −15.6581 | −18.6828 |
| 8132318 | ANLN | NM_018685 | −64.8133 | −20.2137 | −28.6235 |
| 7900699 | CDC20 | NM_001255 | −30.2129 | −16.2126 | −19.2514 |
| 8108301 | KIF20A | NM_005733 | −57.8542 | −23.8555 | −34.2476 |
| 8149955 | PBK | NM_018492 | −32.5616 | −19.3474 | −20.0843 |
| 7982889 | NUSAP1 | NM_016359 | −22.5633 | −20.4159 | −22.6629 |
| 8095585 | SLC4A4 | NM_001098484 | −22.6649 | −18.7589 | −17.6102 |
| 7979307 | DLGAP5 | NM_014750 | −33.3888 | −29.7461 | −24.9006 |
| 7923086 | ASPM | NM_018136 | −42.0298 | −38.6657 | −27.6311 |

TABLE 9
Gene expression profiles for samples from three individuals (identified by
numbers 731, 970, and 1650) of hepatic differentiated (day 38) vs. undifferentiated
cell types. All genes displaying at least 15-fold up- or down-regulation common
in all three individuals are shown.

| | | | | Fold- | |
|---|---|---|---|---|---|
| Probe | | mRNA | Fold-Change | Change | Fold-Change |
| Set ID | Gene Symbol | Accession | (731) | (970) | (1650) |
| 8092970 | APOD | NM_001647 | 60.8751 | 50.9398 | 30.225 |
| 8162394 | ASPN | NM_017680 | 42.8124 | 29.1902 | 43.6651 |
| 7914342 | FABP3 | NM_004102 | 29.3423 | 40.2438 | 21.7561 |
| 7909568 | DTL | NM_016448 | −24.2825 | −27.2865 | −18.1673 |
| 8130578 | SNORA20 | NR_002960 | 39.1874 | 31.3684 | 52.5595 |
| 8135909 | LEP | NM_000230 | 31.0359 | 40.9881 | 16.5296 |
| 7938329 | SNORA23 | NR_002962 | 17.3311 | 17.0651 | 28.392 |
| 7916898 | DEPDC1 | NM_001114120 | −22.0796 | −33.1562 | −24.0738 |
| 8100154 | CORIN | NM_006587 | 23.4492 | 54.0982 | 25.15 |
| 8097356 | PLK4 | NM_014264 | −15.5121 | −22.1399 | −15.9111 |
| 8040223 | RRM2 | NM_001165931 | −19.3484 | −17.863 | −16.6101 |
| 8142981 | PODXL | NM_001018111 | −38.9625 | −46.781 | −30.0028 |
| 7970513 | SKA3 | NM_145061 | −20.425 | −28.1759 | −21.8384 |
| 7989647 | KIAA0101 | NM_014736 | −19.6187 | −17.0796 | −35.7409 |
| 8001133 | SHCBP1 | NM_024745 | −34.4058 | −26.5433 | −22.0365 |
| 8117594 | HIST1H2BM | NM_003521 | −36.9067 | −32.7559 | −42.5919 |
| 8145570 | ESCO2 | NM_001017420 | −37.2988 | −40.5672 | −41.1984 |
| 7927710 | CDK1 | NM_001786 | −26.9834 | −25.503 | −32.2489 |
| 8094278 | NCAPG | NM_022346 | −23.6391 | −39.5451 | −25.4224 |
| 8046380 | ITGA6 | NM_000210 | −24.2236 | −40.342 | −23.6123 |
| 7929334 | CEP55 | NM_018131 | −25.3772 | −28.7031 | −25.4387 |
| 8054702 | CKAP2L | NM_152515 | −15.3705 | −31.0795 | −20.9151 |
| 8124388 | HIST1H3B | NM_003537 | −18.1735 | −25.0438 | −15.967 |
| 7982757 | CASC5 | NM_170589 | −27.5426 | −38.8728 | −28.0519 |
| 8061579 | TPX2 | NM_012112 | −20.4154 | −23.7366 | −25.4272 |
| 7937020 | MKI67 | NM_002417 | −21.878 | −23.1598 | −31.4197 |
| 7974404 | CDKN3 | NM_005192 | −23.4596 | −33.8414 | −30.4806 |
| 7906930 | NUF2 | NM_145697 | −26.5613 | −38.0888 | −15.9209 |
| 8120838 | TTK | NM_003318 | −28.9412 | −35.1878 | −23.46 |
| 7983969 | CCNB2 | NM_004701 | −23.4382 | −30.5261 | −26.8686 |
| 8054580 | BUB1 | NM_004336 | −22.0707 | −28.1837 | −21.9585 |
| 7929258 | KIF11 | NM_004523 | −26.3799 | −37.062 | −19.1903 |
| 7909708 | CENPF | NM_016343 | −15.913 | −27.3463 | −19.3341 |
| 8014974 | TOP2A | NM_001067 | −24.6813 | −33.8132 | −25.894 |
| 8132318 | ANLN | NM_018685 | −43.6733 | −48.0524 | −59.9009 |
| 7900699 | CDC20 | NM_001255 | −26.1873 | −22.3527 | −20.3885 |
| 8108301 | KIF20A | NM_005733 | −42.9814 | −52.6275 | −55.453 |
| 8149955 | PBK | NM_018492 | −21.2251 | −27.3684 | −30.4638 |
| 7982889 | NUSAP1 | NM_016359 | −22.6107 | −25.0294 | −24.9593 |
| 7994109 | PLK1 | NM_005030 | −17.9671 | −19.9368 | −15.4117 |
| 7979307 | DLGAP5 | NM_014750 | −30.8046 | −58.6434 | −35.4778 |
| 7923086 | ASPM | NM_018136 | −34.9205 | −37.761 | −44.1499 |
| 7976567 | BDKRB1 | NM_000710 | −24.9668 | −34.2236 | −24.0507 |

TABLE 10
Gene expression profiles for samples from three individuals (identified by
numbers 731, 970, and 1650) of hepatic differentiated (day 45) vs. undifferentiated
cell types. All genes displaying at least 15-fold up- or down-regulation common
in all three individuals are shown.

| | | | | Fold- | |
|---|---|---|---|---|---|
| Probe | | mRNA | Fold-Change | Change | Fold-Change |
| Set ID | Gene Symbol | Accession | (731) | (970) | (1650) |
| 8162394 | ASPN | NM_017680 | 66.373 | 35.995 | 39.7561 |
| 7920873 | SNORA42 | NR_002974 | 52.5153 | 16.6643 | 45.3536 |
| 7914342 | FABP3 | NM_004102 | 18.8799 | 36.3798 | 22.5329 |
| 7909568 | DTL | NM_016448 | −41.9387 | −31.0817 | −21.635 |
| 8130578 | SNORA20 | NR_002960 | 58.1453 | 41.5835 | 74.3977 |
| 8135909 | LEP | NM_000230 | 34.2576 | 53.2549 | 20.2672 |
| 8023392 | SNORA37 | NR_002970 | 26.9879 | 15.05 | 31.1647 |
| 7938329 | SNORA23 | NR_002962 | 28.8382 | 22.8245 | 36.8451 |
| 8062490 | SNORA60 | NR_002986 | 60.8326 | 22.1889 | 58.1912 |
| 7916898 | DEPDC1 | NM_001114120 | −24.6832 | −23.8046 | −34.9363 |
| 8100154 | CORIN | NM_006587 | 25.0571 | 50.6858 | 17.613 |
| 7982058 | SNORD115- | NR_003343 | 36.6807 | 16.2073 | 36.5537 |
| | 26 | | | | |
| 8040223 | RRM2 | NM_001165931 | −30.9139 | −18.4384 | −17.9048 |
| 8142981 | PODXL | NM_001018111 | −50.2289 | −37.483 | −34.4396 |
| 7970513 | SKA3 | NM_145061 | −25.1129 | −17.3807 | −19.4056 |
| 7989647 | KIAA0101 | NM_014736 | −32.6604 | −22.5218 | −21.7481 |
| 8001133 | SHCBP1 | NM_024745 | −32.5727 | −22.496 | −20.6552 |
| 8117594 | HIST1H2BM | NM_003521 | −46.5603 | −45.8284 | −37.353 |
| 8145570 | ESCO2 | NM_001017420 | −33.1182 | −32.1434 | −25.9359 |
| 7927710 | CDK1 | NM_001786 | −25.5392 | −37.6064 | −34.6591 |
| 8094278 | NCAPG | NM_022346 | −19.769 | −29.0313 | −18.8391 |
| 7960340 | FOXM1 | NM_202002 | −16.4606 | −18.5237 | −17.1505 |
| 8046380 | ITGA6 | NM_000210 | −32.5615 | −30.1129 | −23.3636 |
| 7929334 | CEP55 | NM_018131 | −32.0093 | −32.2956 | −24.1042 |
| 8054702 | CKAP2L | NM_152515 | −16.893 | −22.3094 | −20.0645 |
| 8085754 | SGOL1 | NM_001012410 | −18.9859 | −34.3683 | −20.8895 |
| 8124388 | HIST1H3B | NM_003537 | −27.191 | −24.8563 | −20.42 |
| 7982757 | CASC5 | NM_170589 | −34.8038 | −40.8005 | −34.4483 |
| 8061579 | TPX2 | NM_012112 | −31.1833 | −21.2525 | −32.1726 |
| 7937020 | MKI67 | NM_002417 | −25.4988 | −25.0222 | −19.6793 |
| 7974404 | CDKN3 | NM_005192 | −31.3203 | −34.6756 | −26.6261 |
| 7906930 | NUF2 | NM_145697 | −23.724 | −20.8188 | −19.014 |
| 8120838 | TTK | NM_003318 | −32.3815 | −26.2418 | −22.913 |
| 7983969 | CCNB2 | NM_004701 | −28.6658 | −38.6225 | −27.782 |
| 8054580 | BUB1 | NM_004336 | −30.4457 | −28.3179 | −23.9106 |
| 7929258 | KIF11 | NM_004523 | −16.3365 | −25.3002 | −29.3944 |
| 7909708 | CENPF | NM_016343 | −19.5955 | −15.9155 | −21.2705 |
| 8102643 | CCNA2 | NM_001237 | −17.4267 | −17.7815 | −15.8523 |
| 8014974 | TOP2A | NM_001067 | −23.8265 | −27.5626 | −26.1444 |
| 8132318 | ANLN | NM_018685 | −50.3464 | −36.3315 | −64.557 |
| 7900699 | CDC20 | NM_001255 | −41.9813 | −24.4716 | −18.2385 |
| 8108301 | KIF20A | NM_005733 | −43.3796 | −49.1219 | −68.7341 |
| 8149955 | PBK | NM_018492 | −25.7263 | −30.8167 | −18.0083 |
| 7982889 | NUSAP1 | NM_016359 | −21.9692 | −31.7755 | −25.5242 |
| 7979307 | DLGAP5 | NM_014750 | −40.3228 | −39.0419 | −32.3877 |
| 7923086 | ASPM | NM_018136 | −57.0446 | −47.2426 | −49.2828 |
| 7976567 | BDKRB1 | NM_000710 | −20.3252 | −30.9189 | −22.9959 |

While the present invention has been disclosed with reference to certain aspects and embodiments, persons of ordinary skill in the art will appreciate that numerous modifications, alterations, and changes to the described aspects are possible without departing from the sphere and scope of the present invention. Accordingly, it is intended that the present inventions not be limited to the described aspects and embodiments described herein, but that the inventions be understood consistent with the full spirit and scope in which they are intended to be understood, including equivalents of the particular aspects and embodiments described herein.

What is claimed is:

1. A method of generating a 3-dimensional tissue engineering model comprising the steps of:
(a) propagating multipotent stem cells from human skin fibroblast culture by growing the cells in a culture containing amniotic fluid growth medium (AFM) and allowing the cells to propagate for at least 3 passages; and
(b) subjecting said multipotent stem cells to lineage-specific differentiation by culturing said multipotent stem cells in cells in a culture setting that will foster 3-dimensional tissue growth, such as a scaffold or matrix.

2. The method of claim 1, wherein said culture further comprises Embryonic Cell Qualified Fetal Bovine Serum (ES-FBS).

3. The method of claim 2, wherein the cells are subject to at least 3, 4, 5, 6, 7, or 8 passages in culture.

4. The method of claim 3, further comprising the step of determining the number of multipotent stem cells in the culture.

5. The method of claim 4, wherein the number of CD117⁺ multipotent stem cells in the culture can be determined after each passage.

6. The method of claim 5, wherein the human skin fibroblast culture is prolonged by continued passages in the culture until a high number of CD117⁺ multipotent stem cells is attained.

7. The method of claim 6, wherein the propagated CD 117⁺ multipotent stem cells are subject to differentiation when the CD117⁺ cell count reaches at least about 85%.

8. The method of claim 7, wherein the propagated cells are cryopreserved after step (a) but before step (b).

9. The method of any of claims 1-8, wherein the propagated multipotent stem cells are capable of differentiating into any of the three germ layers.

10. The method of claim 9, wherein the propagated multipotent stem cells are capable of differentiation into adipose, hepatic, muscle, or nerve cells under suitable culture conditions.

11. The method of claim 10, wherein the suitable culture conditions are conditions will foster 3-dimensional tissue growth are culture plates containing laminin-coated beads.

12. The method of claim 11, wherein the culture plates containing laminin-coated beads are created by:
(a) dissolving laminin in cold phosphate buffer saline (PBS) placed in a tissue culture plate;
(b) adding sterile spherical glass beads or a mix of spherical glass beads to the laminin;
(c) placing the culture plate in an incubator at 37° C. for at least 12 hours in order to induce polymerization of laminin; and
(d) removing excess PBS and allowing the culture plate to completely air dry.

13. A method of generating a 3-dimensional tissue engineering model comprising the steps of:
(a) propagating multipotent stem cells from human skin fibroblast culture by growing the cells in a culture containing amniotic fluid growth medium (AFM) and allowing the cells to propagate for at least 3 passages;
(b) culturing the multipotent stem cells in the laminin-coated bead plates in a tissue culture media that promotes differentiation into one of the three germ layers,
wherein the laminin-coated bead plates were created by:
(1) dissolving laminin in cold phosphate buffer saline (PBS) placed in a tissue culture plate;
(2) adding sterile spherical glass beads or a mix of spherical glass beads to the laminin;
(3) placing the culture plate in an incubator at 37° C. for at least 12 hours in order to induce polymerization of laminin;
(4) removing excess PBS and allowing the culture plate to completely air dry;
(5) adding the multipotent stem cells to the laminin-coated bead plates; and
(6) plating the multipotent stem cells in the laminin-coated bead plates with the multipotent stem cells in an incubator at 37° C.; and
(c) subjecting the multipotent stem cells to lineage-specific differentiation under suitable conditions into cells of any of three germ layers.

14. A method for identifying one or more genes involved in the process of lineage-specific differentiation, said method comprising the steps of:
(a) propagating multipotent stem cells from human skin fibroblast culture by growing the cells in a culture containing amniotic fluid growth medium (AFM) and allowing the cells to propagate for at least 3 passages;
(b) subjecting said multipotent stem cells to lineage-specific differentiation by culturing said multipotent stem cells under culture conditions suitable for lineage-specific differentiation until differentiated cells result;
(c) subjecting said differentiated cells to gene expression profiling using microarray technology; and
(d) determining which one or more genes is upregulated or downregulated during the process of lineage-specific differentiation.

15. The method of claim 14, wherein said culture containing amniotic fluid growth medium (AFM) further comprises Embryonic Cell Qualified Fetal Bovine Serum (ES-FBS).

16. The method of claim 15, wherein the cells are subject to at least 3, 4, 5, 6, 7, or 8 passages in culture.

17. The method of claim 16, further comprising the step of determining the number of multipotent stem cells in the culture.

18. The method of claim 17, wherein the number of CD117⁺ multipotent stem cells in the culture can be determined after each passage.

19. The method of claim 18, wherein the human skin fibroblast culture is prolonged by continued passages in the culture until a high number of CD117⁺ multipotent stem cells is attained.

20. The method of claim 19, wherein the propagated CD117⁺ multipotent stem cells are subject to differentiation when the CD117⁺ cell count reaches at least about 85%.

21. The method of claim 20, wherein the propagated cells are cryopreserved after step (a) but before step (b).

22. The method of any of claims 14-21, wherein the propagated multipotent stem cells are capable of differentiating into any of the three germ layers.

23. The method of claim 22, wherein the propagated multipotent stem cells are capable of differentiation into adipose, hepatic, muscle, or nerve cells under suitable culture conditions.

24. The method of claim 23, wherein the suitable culture conditions will foster 3-dimensional tissue growth, such as a scaffold or matrix.

25. The method of claim 24, wherein the culture conditions that will foster 3-dimensional tissue growth are culture plates containing laminin-coated beads.

26. The method of claim 25, wherein the culture plates containing laminin-coated beads are created by:
(a) dissolving laminin in cold phosphate buffer saline (PBS) placed in a tissue culture plate;
(b) adding sterile spherical glass beads or a mix of spherical glass beads to the laminin;
(c) placing the culture plate in an incubator at 37° C. for at least 12 hours in order to induce polymerization of laminin; and
(d) removing excess PBS and allowing the culture plate to completely air dry.

27. An isolated multipotent stem cell, or a collection of culture of isolated multipotent stem cells, obtained by a method of propagating multipotent stem cells from human skin fibroblast culture by growing the cells in a culture containing amniotic fluid growth medium (AFM) and allowing the cells to propagate for at least 3 passages.

28. The isolated multipotent stem cell, or a collection of culture of isolated multipotent stem cells of claim 27, wherein the culture further comprises Embryonic Cell Qualified Fetal Bovine Serum (ES-FBS).

29. The isolated multipotent stem cell, or a collection of culture of isolated multipotent stem cells of claim 28, wherein the multipotent stem cells are capable differentiating into any of the three germ layers.

30. An isolated differentiated cell, or a collection of culture of isolated differentiated cells, obtained by:
(a) propagating multipotent stem cells from human skin fibroblast culture by growing the cells in a culture containing amniotic fluid growth medium (AFM) and allowing the cells to propagate for at least 3 passages; and
(b) subjecting said multipotent stem cells to lineage-specific differentiation by culturing said multipotent stem cells under culture conditions suitable for lineage-specific differentiation until differentiated cells result.

31. The isolated differentiated cell, or a collection of culture of isolated differentiated cells of claim 30, wherein the differentiated cells are cells of any of the three germ layers.

32. The isolated differentiated cell, or a collection of culture of isolated differentiated cells of claim 31, wherein the cells of any of the three germ layers include adipose, hepatic, muscle, or nerve cells.

33. The isolated differentiated cell, or a collection of culture of isolated differentiated cells of any one of claims 30-32, wherein the culture conditions suitable for lineage-specific differentiation foster 3-dimensional tissue growth.

34. The isolated differentiated cell, or a collection of culture of isolated differentiated cells of claim 33, wherein the culture conditions suitable for lineage-specific differentiation foster 3-dimensional tissue growth are culture plates containing laminin-coated beads.

35. The isolated differentiated cell, or a collection of culture of isolated differentiated cells of claim 34, wherein the culture plates containing laminin-coated beads are created by:
(a) dissolving laminin in cold phosphate buffer saline (PBS) placed in a tissue culture plate;
(b) adding sterile spherical glass beads or a mix of spherical glass beads to the laminin;
(c) placing the culture plate in an incubator at 37° C. for at least 12 hours in order to induce polymerization of laminin; and
(d) removing excess PBS and allowing the culture plate to completely air dry.
